(19) United States
(12) Patent Application Publication
(10) Pub. No.: US 2003/0206958 A1
(43) Pub. Date: Nov. 6, 2003

(54) Chitosan biopolymer for the topical delivery of active agents

(76) Inventors: Maurizio V. Cattaneo, Quincy, MA (US); Marie-France Demierre, Quincy (FR)

Correspondence Address: EDWARDS & ANGELL, LLP, P.O. BOX 9169, BOSTON, MA, 02209, US

(21) Appl. No.: 10/221,307

(22) PCT Filed: Dec. 22, 2000
(86) PCT No.: PCT/US00/35319

(51) Int. Cl. A61K 31/7076; A61K 31/573; A61K 31/203; A61K 9/14; A61K 9/16; A61K 9/50; A61K 31/19; A61K 31/07

(52) U.S. Cl. 424/488; 514/55; 514/179; 514/559; 514/725; 514/50; 514/557

(57) ABSTRACT

The present invention relates to a carrier base for the topical delivery of an active agent comprising a high viscosity chitosan biopolymer. The invention further relates to a method of controlling the release of an active agent from a carrier base, comprising as a carrier base a high viscosity chitosan; providing the active agent; and mixing the active agent and the chitosan. Preferably, the carrier base comprises a high viscosity chitosan having a molecular weight of at least about 100,000 Dalton, more preferably at least about 250,000 Dalton and most preferably at least about 300,000 Dalton. In other preferred embodiments the chitosan has a concentration of at least about 2 weight %.

### FIELD OF THE INVENTION

This invention relates to carrier bases for the topical delivery of active agents comprising high viscosity chitosan biopolymers. Preferred carrier bases comprise chitosan having a molecular weight of at least 250,000 Dalton. The invention also relates to carrier bases comprising high viscosity chitosan at a concentration of at least 2 weight%. The present invention further provides a delivery system for therapeutic agents, such as retinolds, that overcomes many of the previously known problems associated with delivery systems for retinoids.

### BACKGROUND OF THE INVENTION

A number of changes occur in skin tissue as a consequence of aging, photodamage, and diseases, e.g., skin cancer and acne. Skin connective tissue is comprised primarily of fibrillar collagen bundles and elastic fibers, along with extracellular matrix (ECM) molecules such as glycosaminoglycans (GAG), proteoglycans, glycoproteins, peptide growth factors. Keratinocytes and fibroblasts are the main cell types embedded within the ECM. The predominant component of the ECM is hyaluronan (HA). HA is the primordial and simplest of the GAGs, and the first ECM to be developed in the developing embryo. HA is thought to be largely a product of fibroblasts.

The components of the extracellular matrix (ECM) form a highly organized structure endowed with hydration properties, and structural proteins such as collagen and to a lesser extent, elastin. HA is the primordial and simplest of the GAGs, and the first ECM to be developed in the developing embryo. HA is thought to be largely a product of fibroblasts.

A number of changes occur in the structure of skin connective tissue as a consequence of aging or photodamage. Age-related changes include a decrease in the number of fibroblasts, and connective tissue abnormalities such as (1) thinning of the collagen fiber bundles, (2) an increase in space between collagen fiber bundles, (3) an increase in collagen fiber bundle disorganization and (4) increase in depth of disorganization (Varani et al., 2000). In addition, the HA in the epidermal extracellular matrix has disappeared completely in aged skin (Neudeker et al., 2000). These alterations are believed to be largely responsible for the thin, fragile, and finely wrinkled quality of naturally-aged skin. Photoaged skin is characterized by the presence of elastotic material and damage to the collagen bundles. Clinically, photoaged skin appears thick and rough, with course wrinkles and mottled pigmentation (Lavker, 1995).

The alterations in skin connective tissue in skin aging and photodamage and skin diseases seem to be mediated mainly by collagen which comprises the bulk of the connective tissue (90% wet weight) and by hyaluronan which is the predominant component of the extracellular matrix. In terms of quantity both reduction in collagen synthesis and increased destruction seem to occur. Collagen synthesis is reduced in both photoaged and naturally aged skin (Griffiths et al., 1993; Talwar et al., 1995; Varani et al., 2000). In vivo studies have demonstrated decreased collagen synthesis in aged fibroblasts (Johnson et al., 1986, Gregory et al. 1986; Mays et al., 1990; Furth, 1991) In photodamaged skin UV irradiation has been shown to increase production of matrix metalloproteinases (MMP) which destroy collagen and cause tissue damage (Fisher et al., 1996, 1997). The quality of the fiber bundle architecture seems to be mediated by extracellular and structural molecules such as hyaluronan.

There are many known agents that are used for the treatment of skin diseases and defects, including, e.g., retinoids, vitamins, and alpha-hydroxy acids. Topical application of retinoids such as All-trans retinoic acid and retinol has been shown to stimulate collagen synthesis in naturally aged as well as photoaged skin (Varani et al., 2000; Griffiths et al., 1993). The active substance seems to be All-trans retinoic acid. However, the two retinoids All-trans retinoic acid and retinol are related. Indirect evidence exists that retinol transforms into All-trans retinoic acid in human skin (Kang et al, 1995). Retinoids appear to affect the quantity of collagen by increasing the number of collagen-producing fibroblasts, increasing collagen synthesis and/or by reducing MMP levels in skin, thereby decreasing destruction of collagen (Varani et al., 2000). However, retinoids do not seem capable of affecting the quality of the collagen being produced as evidenced by no change in the dermal connective tissue abnormalities after retinoid treatment (Varani et al., 2000). For increasing the quality of the collagen being produced by the retinoids there seems to be a need for additional molecules which play a role in tissue reorganization.

Although retinoid treatment induced measurable changes in the dermal fibroblast population, it did not alter age-associated connective tissue abnormalities such as correct collagen fiber deposition (Varani et al., 2000). Thus, it would be desirable to have a carrier base that is capable of altering these abnormalities and reverse or minimize the effects of aging or photodamage on the skin.

Retinoids are also used to treat other skin conditions such as acne, actinic keratosis, psoriasis, skin cancers and have been found to useful therapeutic agents in the chemoprevention of melanoma (Stam-Postuma, 1998; Halpern, 1994; Kligman, 1998).

The incidence of melanoma is increasing in the United States at a rate of about 2.7% annually, even as most other cancers are experiencing a decline in incidence. Furthermore, melanoma is the seventh most commonly diagnosed cancer in U.S. men and women. Chemoprevention is a strategy to prevent the development of melanoma through the administration of drugs. The recognition of dysplastic nevi as markers of melanoma risk and intermediate steps of melanocytic tumor progression has significant implications for melanoma chemoprevention.

The incidence of malignant melanoma of the skin, the most serious form of skin cancer, is increasing faster than that of any other cancer in the United States (Koh 1991). Trends in melanoma incidence rates have continued to increase substantially (from 1990-1996: =2.7% per year; p<0.001) while all other cancer incidence decreased (except for non-Hodgkin's lymphoma) (Wingo et al., 1999). Data from the Surveillance, Epidemiology, and End Results Program Registry (SEER 1973-1994) indicates that the increasing incidence rates of melanoma may represent a true increase in cancer rates with data also showing an increase in advanced disease (thick tumors-2 year mortality). (Dennis, 1999) similar to that reported in Australia (Hall et al.,1999).

While strategies for malignant melanoma have included (1) public health interventions (Koh and Geller, 1998), (2) adjuvant therapies (Demierre and Koh, 1997) and (3) immunotherapy (Curiel-Lewandrowski and Demierre, 1999), recent research suggests chemoprevention is an important strategy for the management of malignant melanoma (Halpern, 1994, 1998). Chemoprevention entails the use of specific agents to block, reverse or suppress carcinogenesis and thereby prevent the development of primary or secondary cancers Melanocytic nevi, particularly dysplastic nevi confer a risk factor for the development of melanoma, with quantitative measures correlating directly with the magnitude of risk. (Tucker et al 1997; Grob et al., 1990; Egan et al., 1998; Meier et al., 1998) and a count of benign melanocytic nevi as a major indicator of risk for non-familial nodular and superficial spreading and nodular melanoma (Grob et al., 1990). In a multicenter prospective case-control study of 716 newly diagnosed melanoma patients and 1014 controls conducted by Tucker et al.(1997), an increased risk of melanoma was determined according to the number of non-dysplastic and dysplastic nevi. Individuals with numerous small nevi had a double risk of melanoma. Having additional large non-dysplastic nevi increases the risk four-fold. Having just one dysplastic nevus was associated with approximately a 2-fold risk, while 10 or more conferred a 12-fold risk of melanoma.

Furthermore, clinical and histopathologic features of melanoma have suggested five steps of melanoma progression: (1) common acquired and congenital nevi with structurally normal melanocytes, (2) dysplastic nevus with structural and architectural atypia, (3) early radial growth phase primary melanoma, (4) advanced vertical growth phase primary melanoma with competence for metastasis, and (5) metastatic melanoma (Sauter and Herlyn, 1998). The recognition of dysplastic nevi both as markers of melanoma risk and intermediate steps of melanocytic tumor progression has significant implication for melanoma chemoprevention.

A national chemoprevention multicenter randomized Phase II trial led by the Eastern Cooperative Oncology Group (ECOG) is investigating the effects of topical tretinoin (ATRA) and systemic fenretinide (4-HPR). Small pilot studies have demonstrated a significant effect of topical tretinoin on the appearance and histology of dysplastic nevi. Topical tretinoin is also active in the treatment of inflammatory diseases (acne vulgaris), precancerous lesions (actinic keratosis) and photodamage.

Retinoids are among the most promising chemopreventive agents with clinical effects of retinoid chemoprevention having been demonstrated in cancers of the head and neck, lung, cervix, ovaries and skin (Lotan, 1996; Sankaranarayanan and Mathew, 1996, Labrecque et al., 1999). Topical application of tretinoin (all-trans retinoic acid, ATRA) has been shown to decrease melanocyte numbers and reduce melanocytic atypia in the treatment of photodamaged skin (Bhawan et al., 1996) and small pilot studies have demonstrated a significant effect of topical tretinoin on the appearance and histology of dysplastic nevi (Halpern et al., 1994, 1998; Stam-Posthuma et al., 1998). In addition, in a malignant melanoma murine model, with ATRA or 9-cis-RA treatment there was a reversible conversion of malignant melanoma into a benign, melanocytic phenotype (Spanjaard et al., 1997; Clifford et al., 1990). It is well known that there are two structurally and pharmacologically distinct families of retinoid receptors: the retinoic acid receptor (RAR) family with subtypes α, β, γ and the retinoid X receptor (RXR) family with subtypes α, β, γ. ATRA binds and activates RARs, whereas the panagonist 9-cis-RA, a novel retinoid, binds and activates all six of the retinoid receptors. Of note, melanoma expresses all three of the RAR subtypes (Nagpal and Chandraratna, 1996). These data suggests that melanoma chemoprevention of persons at high risk of developing melanoma might benefit from both ATRA and 9-cis-RA.

In presently used topical delivery systems for agents used to treat skin ailments, one side effect is increased irritation. For example, compared to oral administration, topical delivery of retinoids increases the concentration of retinoids in the dermal compartment 10- to 100-fold (Lehman et al., 1988). However, topical tretinoin (ATRA) induces irritation in 90% of patients (Gilchrest, 1997), and other side effects include patchy erythema, localized swelling, xerosis, and scaling. Irritation has been attributed, in part, by an overload of the tretinoin dependent pathways with non-physiological amounts of exogenous tretinoin in the skin. (Siegenthaler et al., 1994). This irritation may be the reason for discontinuation of treatment for close to 50% of patients (Stam-Posthuma et al., 1998). This high incidence of irritation, leading to poor compliance, can preclude its use.

The incorporation of drugs into polymeric carriers provides advantages, e.g., preferable tissue distribution of the drug, prolonged half-life, controlled drug release and reduction of drug toxicity. Examples of percutaneous drug delivery systems for retinoids delivery presently on the market include ATRA formulations containing a synthetic material, polyolprepolymer-2 (PP2) (Avita, Penederm Inc., Foster City, Calif.). These retinoid formulations have been shown to be less irritating than currently marketed ATRA formulations (Quigley and Bucks, 1998). The addition of the synthetic polymer appeared to reduce the percutaneous flux to about 50% of an equivalent ATRA commercial formulation (0.025% ATRA) after 6 hours of delivery. Another synthetic polymer system based on acrylates for retinoid delivery is described in U.S. Pat. Nos. 5,145,675 and 5,955,109 in Won et al. (1992; 1999). However, these formulations utilize a non-biodegradable synthetic polymer as a carrier of the drug. High molecular weight polymers (360,000 to 400,000 Dalton) have been shown to penetrate the stratum comeum (Brown et al., 1999). The possibility of other polymers, such as the synthetic polymers described above, to penetrate the skin and enter the systemic circulation has been suggested by the authors after careful radiolabeled analysis of the tissue distribution and accumulation in various tissue organs of their target high molecular weight polymer after topical application (Brown et al., 1999). Thus it would be desirable to have a topical delivery system which is entirely biodegradable due to the likelihood of it entering the systemic circulation and accumulating in target tissues.

In addition, there is presently no controlled topical delivery system of retinoids for use in melanoma chemoprevention. A controlled delivery system could make retinoid topical therapy a viable chemoprevention treatment for melanoma. In addition, it would be useful to have a delivery system that utilizes a non-synthetic carrier which is biodegradable after penetrating the skin layers.

Thus, it would be desirable to have a controlled delivery vehicle for active agents used to treat skin ailments, which would prevent the irritation seen in present treatments. For example such a delivery system for retinoids would enable chronic use of topical retinoids for treating skin ailments, including for melanoma chemoprevention. A controlled delivery system could make tretinoin topical therapy a viable chemoprevention treatment for melanoma in individuals with dysplastic nevi who are at high risk of developing melanoma.

Chitosan is a natural, biodegradable cationic polysaccharide derived by deacetylating chitin, a natural material extracted from fungi, the exoskeletons of shellfish and from algae and has previously been described as a promoter of wound healing (Balassa, 1972; Balassa, 1975). Chitosan comprises a family of polymers with a high percentage of glucosamine (normally 70-99%) and N-acetylated glucosamine (1-30%) forming a linear saccharide chain of molecular weight from 10,000 up to about 1000,000 Dalton. Chitosan is polycationic. Chitosan, through its cationic glucosamine groups, interacts with anionic proteins such as keratin in the skin conferring bioadhesive characteristics. When not deacetylated, the acetamino groups of chitosan are an interesting target for hydrophobic interactions and contribute to some degree to its bioadhesive characteristics. Modified chitins and chitosans have been administered to humans in the form of dressings for wounded soft tissues and for the controlled delivery of drugs (Muzzarelli et al, 1986; 1999; Muzzarelli, 1993; 1996; Tokura and Azuma, 1992; Wada, 1995; Maekawa and Wada, 1990; Mita et al., 1989). For the purpose of soft tissue healing the most relevant characteristics of chitin-based biomaterials are their biodegradability, biocompatibility and similarity to hyaluronan, beside their capacity to release glucosamine and N-acetyl-glucosamine monomers and oligomers (Muzzarelli, 1999).

Chitosan is insoluble in neutral to alkaline water and thus, it has to be exposed to acidic conditions to render it soluble. Methods for solubilizing chitosan include the use of a slightly acid solution (pH<6) containing acidic acid, glycolic acid, lactic acid, or other alpha-hydroxy acids. Other methods include producing derivatives of chitosan which obviate the need for acids to solubilize chitosan. For example, U.S Pat. No. 3,953,608 in Vanlerberghe and Sebag describes a method of making chitosan soluble in water at pH>7 by acylation of the chitosan using organic anhydrides. This patent describes the use of these derivatives mainly as film formers for coloring of the skin, deodorizing products and making antispot products. U.S. Pat. Nos. 4,929,722 and 4,946,870 describe the use of chitosan derivatives in delivery systems for the delivery of pharmaceutical or therapeutic compositions. U.S. Pat. No. 4,929,722 describes, in particular, the method of making a chitin or chitosan salt or covalent derivative from highly crystalline, partially deacetylated chitin or chitosan. TheseF ionic derivatives of chitosan called chitosonium polymers and covalent chitosan derivatives have been made by dispersing chitosan in an aqueous/solvent mixture. U.S. Pat. No. 4,946,870 describes the use of these chitosonium polymers and covalent chitosan derivatives. U.S. Pat. No. 5,300,494 describes the same delivery system to deliver quaternary and related compounds.

It would be useful to have a delivery system that incorporates drugs, such as retinoids, into polymeric carriers to provide advantages such as preferable tissue distribution of the drug, prolonged half life, controlled drug release and reduction of drug toxicity. The use of a controlled topical delivery vehicle for retinoids may prevent the overload of retinoids into the systemic circulation, which may be responsible for irritation and allow chronic use of topical retinoids. In addition, it would be useful to have a controlled topical delivery system of retinoids for melanoma chemoprevention. A controlled delivery system could make tretinoin topical therapy a viable chemoprevention treatment for melanoma.

It would also be useful to have a controlled delivery system for the delivery of retinoids in which the carrier of the drug promotes connective tissue abnormalities in the damaged tissue, in order to increase the effectiveness of the treatment.

### SUMMARY OF THE INVENTION

The present invention relates to a carrier base for the topical delivery of an active agent comprising a high viscosity chitosan biopolymer. Preferably, the carrier base comprises a high viscosity chitosan having a molecular weight of at least about 100,000 Dalton, more preferably at least about 250,000 Dalton and most preferably at least about 300,000 Dalton. In other preferred embodiments the chitosan has a concentration of at least about 2 weight %. In an especially preferred embodiment, the carrier bases comprises a high viscosity chitosan biopolymer having a molecular weight of at least about 300,000 Dalton and at a concentration of at least 2 weight %.

The present invention also relates to a composition for the topical delivery of an active agent comprising a carrier base as described above and an active agent. Examples of active agents include pharmaceutical actives and therapeutic actives. Preferred pharmaceutical actives are those used for the treatment of skin diseases, e.g., retinoids, corticosteroids, non-steroidal anti-inflammatory drugs (NSAIDS), hormones, anti-fungal agents, anti-septic agents, local anaesthetics, kerolytic agents, and 5-FU. Examples of useful therapeutic actives include, but are not limited to vitamins and moisturizing agents such as alpha-hydroxy acids, etc. as further described below. In certain embodiments, the compositions contain more than one active agent, thus the compositions comprise at least one additional active agent, which can be either a pharmaceutical active or a therapeutic active. A preferred composition comprises the carrier, retinoids and alpha-hydroxy acid.

In certain compositions of the present invention the chitosan has a molecular weight of at least about 300,000 Daltons. In certain of these embodiments, the chitosan is present in a concentration greater than about 2%. These compositions are especially useful for obtaining the slow, sustained release of the active agent.

In certain embodiments of the present invention, the chitosan has a molecular weight of about 10,000 to about 250,000 Dalton. In certain of these embodiments the chitosan is present in a concentration greater than about 5%, more preferably between about 5% up to about 8%.

The invention further relates to compositions for the topical delivery of retinoids comprising a carrier base and a retinoid, wherein the carrier base comprises a high viscosity chitosan. Preferably, the carrier base comprises a high viscosity chitosan having a molecular weight of at least about 100,000 Dalton, more preferably at least about 250,000 Dalton and most preferably at least about 300,000 Dalton. In other preferred embodiments the chitosan has a concentration of at least about 2 weight %. In an especially preferred embodiment, the carrier bases comprises a high viscosity chitosan biopolymer having a molecular weight of at least about 300,000 Dalton and at a concentration of at least 2 weight %.

The invention provides for compositions of the present invention in the form of gels, creams and lotions. The manufacture of such gels, creams or lotions are known in the art.

The invention further relates to a method of controlling the release of an active agent from a carrier base, comprising as a carrier base a high viscosity chitosan; providing the active agent; and mixing the active agent and the chitosan. Preferably, the carrier base comprises a high viscosity chitosan having a molecular weight of at least about 100,000 Dalton, more preferably at least about 250,000 Dalton and most preferably at least about 300,000 Dalton. In other preferred embodiments the chitosan has a concentration of at least about 2 weight %. In an especially preferred embodiment, the carrier base comprises a high viscosity chitosan biopolymer having a molecular weight of at least about 300,000 Dalton and at a concentration of at least 2 weight %.

In certain methods, the method further comprises the step of selecting a concentration of chitosan depending on the molecular weight of the chitosan provided so that a viscosity of at least about 100 cps is obtained.

In preferred methods of controlling the release of an active agent from a carrier, the active agent comprises a pharmaceutical active, e.g., an agent that is used for the treatment of skin diseases. Examples of pharmaceutical actives include, but are not limited to retinoids, such as corticosteroids, non-steroidal anti-inflammatory drugs (NSAIDS), hormones, antiviral, anti-histamines, anti-fungal agents, anti-septic agents, local anaesthetics, kerolytic agents, 5-FU, etc. In other embodiments, the active agent comprises a therapeutic active, e.g., vitamins, moisturizing agents such as alpha-hydroxy acids, etc., as further described below. In certain embodiments, the composition contains more than one active agent, thus the compositions comprise at least one additional active agent, which can be either a pharmaceutical active or a therapeutic active.

The invention also relates to a method of treating skin diseases providing to the diseased skin a composition containing a high viscosity chitosan biopolymer and an active agent. Preferably, the high viscosity chitosan has a molecular weight of at least about 100,000 Dalton, more preferably at least about 250,000 Dalton and most preferably at least about 300,000 Dalton. In other preferred embodiments the chitosan has a concentration of at least about 2 weight %. In an especially preferred embodiment, the high viscosity chitosan biopolymer has a molecular weight of at least about 300,000 Dalton and at a concentration of at least 2 weight %.

Examples of skin diseases include, but are not limited to, acne, melanoma, premature skin aging, and photodamage. In preferred embodiments the active agent comprises a pharmaceutical active, e.g., an agent that is used for the treatment of skin diseases. Examples of pharmaceutical actives include, but are not limited to retinoids, such as corticosteroids, non-steroidal anti-inflammatory drugs (NSAIDS), hormones, anti-viral, anti-histamines, anti-fungal agents, anti-septic agents, local anaesthetics, kerolytic agents, 5-FU, etc. In other embodiments, the active agent comprises a therapeutic active, e.g., vitamins, moisturizing agents such as alpha-hydroxy acids, etc., as further described below. In certain embodiments, the compositions contains more than one active agent, thus the compositions comprises at least one additional active agent, which can be either a pharmaceutical active or a therapeutic active. In certain embodiments of the present invention, the methods of treating skin diseases comprises the compositions of the present invention, as described herein, in conjunction with other treatments for the disease. For example, in treating precancerous skin conditions, it may be useful to use the compositions of the present invention with standard treatments that use an anti-cancer drug, e.g., 5-FU for the treatment of actinic keratosis.

The invention further relates to compositions for the topical delivery of an active agent comprising a chitosan biopolymer and the active agent, wherein the chitosan has a molecular weight of at least about 300,000 Daltons and is present at a concentration less than about 2%, preferably less than about 1 weight %. These compositions are useful for increasing the transdermal delivery of the active agent.

In preferred compositions of the present invention, the chitosan biopolymer comprises a chitosan having a molecular weight of at least about 100,000 dalton. Preferably the chitosan has a molecular weight ranging from about 250,000 daltons to about 1000,000, more preferably about 300,000 to about 1000,000, and most preferably from about 300,000 to about 800,000 Dalton.

In certain embodiments the chitosan has a molecular weight from about 300,000 to about 800,000, at a concentration of at least about 2%. In other embodiments, the chitosan has a molecular weight from about 100,000 Daltons to about 300,000 and a concentration of at least about 5%.

In preferred methods and compositions of the present invention, the chitosan has a degree of deacetylation of from about 70% to about 90%.

In preferred embodiments, the pharmaceutical active comprises a retinoid. Examples of retinoids comprise retinoic acid or retinol. In preferred embodiments of the present invention, the retinoic acid comprises all trans retinoic acid (ATRA).

### BRIEF DESCRIPTION OF THE DRAWINGS

FIG. 1 is a graph that shows ATRA distribution with chitosan topical delivery.

FIG. 2 shows the use of high molecular weight (HMW) chitosan to enhance transdermal delivery.

FIG. 3 shows ATRA distribution using 3% HMW chitosan.

FIG. 4 is a graph showing ATRA permeation with the high molecular weight chitosan (TD012).

FIG. 5 is a graph that shows ATRA permeation of the high molecular weight chitosan and middle molecular weight chitosan (TM761).

FIG. 6 shows the stability of ATRA gels of the present invention at 20° C.

FIG. 7 shows the stability of retinol creams of the present invention at 40° C.

FIG. 8 shows the stability of ATRA in HMW chitosan.

FIG. 9 is a graph that shows that as the chitosan concentration increases from 1% to 3% this results in a more gradual release of retinoic acid from the chitosan matrix.

### DETAILED DESCRIPTION OF THE INVENTION

The methods of the present invention provide a system of incorporating active agents, e.g., pharmaceuticals, such as retinoids, into polymeric carriers to provide advantages, such as preferable tissue distribution of the drug, prolonged half life, controlled drug release and reduction of drug toxicity. More particularly, the present invention relates to the use of a chitosan carrier for the topical delivery of an active agent, e.g., retinoids, where the sustained release of theedrug can be altered by varying the properties of the chitosan that is used as a carrier base for the drug.

As used herein, the term “active agent” refers to any substance that when introduced into the body has an affect on either the appearance of tissue to which it is applied, or alters the way the body functions. The term “pharmaceutical active” refers to a drug, i.e., a substance which when applied to, or introduced into the body, alters in some way body functions, e.g., altering cell processes. Examples of pharmaceutical actives include, but are not limited to, agents that are used for the treatment of skin diseases, e.g., retinoids, corticosteroids, non-steroidal anti-inflammatory drugs (NSAIDS), hormones, anti-viral agents, anti-fungal agents, anti-septic agents, local anaesthetics, anti-histamines, kerolytic agents, 5-FU, etc. Other examples of such actives include, but are not limited to growth factors, recombinant human interleukin-2 and DNA, RNA and oligonucleotides and the like.

The term “therapeutic active” as used herein, refers to a substance which either alters processes within the body, or alters the cosmetic appearance of the tissue of interest, e.g., skin, but is not technically considered a drug. Examples of therapeutic actives include, but are not limited to, vitamins, e.g., vitamins A, B, C, D and E, alpha-hydroxy acids, moisturizers and other additives, as further described below.

In certain embodiments, the compositions contains more than one active agent, thus the compositions comprises at least one additional active agent, which can be either a pharmaceutical active or a therapeutic active. For example, in a preferred embodiment, the compositions includes a retinoid as a pharmaceutical active and alpha-hydroxy acid as a therapeutic active.

The invention will be discussed in relation to retinoids. However, it is to be understood that any active agent that can be used in a topical delivery system can be used in the compositions and methods of the present invention. Preferably the active agent is a substance that has a molecular weight less than about 300,000 Daltons. For example, preferred agents include retinoids, e.g., retinoic acid and retinol (Vitamin A), 5-FU, anti-fungal agents, anti-viral agents, anti-histamines, hormones and corticosteroids.

The term “topical” as used herein is known in that art and includes the application of the compounds of the present invention to skin surfaces, including mucosal surfaces, such as labial, rectal and genital mucosal surfaces.

The term “carrier base” as used herein includes a component of the delivery system that assists in the release of the active agent that is being delivered. Preferred carrier bases comprise a high viscosity chitosan having a molecular weight of at least about 100,000 Dalton, more preferably at least about 250,000 Dalton and most preferably at least about 300,000 Dalton. In other preferred embodiments the chitosan has a concentration of at least about 2 weight %. In an especially preferred embodiment, the carrier bases comprises a high viscosity chitosan biopolymer having a molecular weight of at least about 300,000 Dalton and at a concentration of at least 2 weight %.

The term “high viscosity” chitosan refers to a chitosan biopolyrner having a viscosity of at least about 100 cps. The viscosity of the chitosan solution can readily be determined by one of ordinary skill in the art, e.g., by the methods described in Li et al., Rheological Properties of aqueous suspensions of chitin crystallites. J Colloid Interface Sc 183:365-373, 1996. In addition, viscosity can be estimated according to Philipof's equation: V=(1+KC)⁸, where V is the viscosity in cps, K is a constant, C is the concentration expressed as a fraction (Form No. 198-1029-997GW, Dow Chemical Company). In certain embodiments, the high viscosity chitosan preferably has a viscosity greater than at least 100 pcs, and more preferably greater than at least 500 cps. The term “low viscosity” chitosan refers to a chitosan solution having a viscosity of at least about 1-30 cps. “Middle viscosity” refers to a chitosan having a viscosity of about 30-100 cps. Viscosity measurements reported here refer to a chitosan solution at 1% concentration in 1% acetic acid measured in a Brookfield LVT viscometer with appropriate spindle at 30 RPM, as common in the art.

The term “high concentration” as used herein, may refer to a concentration of greater than about 2% chitosan in the solution. The term “low concentration” refers to up to about 1% chitosan. The term “middle concentration” refers to between about 1 and about 2%.

The term “high molecular weight” chitosan, also referred to herein as HMW, refers to chitosan having a molecular weight of at least about 250,000 Dalton. The term “middle molecular weight” chitosan, also referred to herein as MMW, refers to chitosan having a molecular weight of at least about 50,000 up to about 250,000 Dalton. The term “low molecular weight” chitosan, also referred to herein as LMW, refers to chitosan having a molecular weight up to about 50,000 Dalton. In preferred embodiments, the carrier base is a chitosan having a molecular weight of at least about 250,000 Dalton, more preferably at least about 300,000.

The compositions and methods of the present invention rely on the discovery of the inventors that the desired viscosity of the chitosans can be achieved by manipulating the concentration, i.e., percentage, of different molecular weight chitosans. For example, as shown in Table 1, a viscosity of greater than 100,000 cps can be obtained by using 12% of a LMW chitosan, 5% of a MMW chitosan or 3% of a HMW chitosan.

TABLE 1
Viscosity-concentration relationship for
different viscosity-grade Chitosans

| | | |
|---|---|---|
| LMW | MMW | HMW |

| Viscosity | | Viscosity | | Viscosity | |
|---|---|---|---|---|---|
| (cps) | % | (cps) | % | (cps) | % |
| 7 | 1 | 66 | 1 | 552 | 1 |
| 21,263 | 9 | 151,403 | 5 | 15,862 | 2 |
| 116,882 | 12 | 3.27 E+06 | 8 | 171,163 | 3 |

The methods and compositions of the present invention enable the control of the active agent by varying the concentration, molecular weight and, therefore the viscosity of the chitosan. For example, in one embodiment of the present invention, the use of a greater concentration of a lower molecular weight chitosan will provide similar release rates as a higher molecular weight chitosan.

Retinoids, e.g., retinoic acid, are hydrophobic and highly insoluble. We have found that delivery of retinoic acid is highly dependent on the viscosity of the carrier base. Thus, we have found that the higher the viscosity of the colloidal solution of chitosan, the slower the release of the agent being delivered. For example, the retinoids in the present compositions are released as the polymer film on the skin surface becomes hydrated. As the film containing the drug and carrier dissolves away, new layers of the compositions containing the drug are exposed, leading to further release of the drug to the affected area of the skin.

The inventors have found that the chitosan-based controlled delivery system of the present invention for delivery of retinoids enhances the transdermal delivery of retinoids where warranted, yet prevents the overload that results from traditional retinoid treatments and thus reduce skin irritation. As discussed further below, experiments using Franz diffusion cells have shown that carrier bases of the present invention slow down the release of retinoids which is delivered across the epidermal membrane, thus limiting the overload of retinoids to the dermal compartment. Thus, the compositions of the present invention enable the slow, sustained release of the drugs, as desired.

The cumulative All-Trans-Retinoic Acid (ATRA) levels in each skin compartment of hairless mouse skin after about 200 hrs exposure to different chitosan formulations is shown in FIG. 1. By varying the viscosity of the chitosan from 550 cps for the 1% High Molecular Weight (HMW) chitosan (MW˜360,000 Dalton) to an estimated 3.27 million cps for the 8% Middle Molecular Weight (MMW) chitosan (MW˜120,000 Dalton) it is possible to obtain a wide range of retinoid distributions. The cumulative percutaneous penetration across the skin is inversely proportional to the amount of retinoid remaining on the skin surface. As the amount of retinoid remaining on the skin surface decreases from around 90% of the applied dose for the 8% MMW chitosan to less than 30% for the 1% HMW, the percutaneous penetration of retinoid increases from less than 10% to around 70%. Likewise, the amount of retinoids in the skin layers increases from less than 1% for the 8% MMW to around 5% for the 1% HMW.

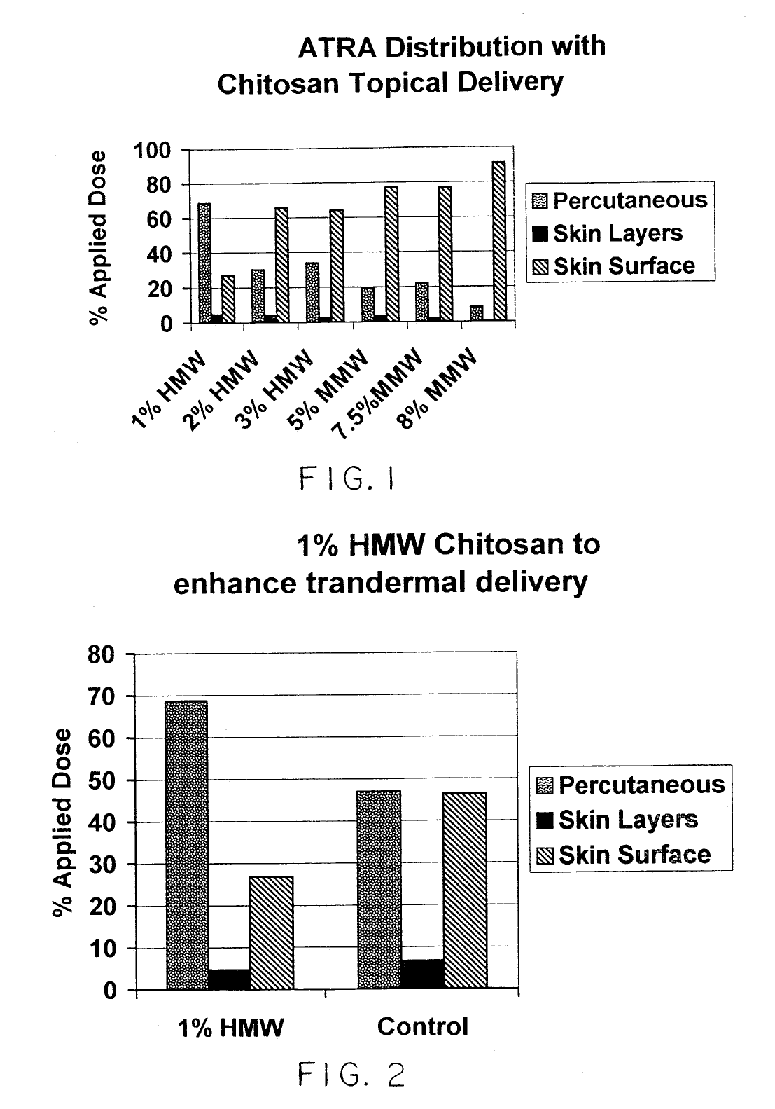

FIG. 2 shows the 1% HMW chitosan, containing 0.1% ATRA compared to a control gel, containing 0.1 g ATRA. The 1% HMW chitosan contains 0.1% ATRA (0.1 g ATRA, 0.04 g butylated hydroxytoluene (BHT), 1 g of Cremophors® RH40, 15 g ethanol (200 proof, 1 g of Chitosan HMW, 81.8 g water, 1 g of glacial acetic acid]. The control gel contained the following: 0.1 g ATRA, 0.04 g BHT, 1 g of Cremophor® RH40, 15 g of ethanol, 0.5 g of Carbopol 940 NF, 76 g water and 0.7 g Triethanolamine The results show a higher percutaneous penetration was obtained with the 1% HMW compared with the standard gel. A full 70% of the applied retinoid. dose was delivered transcutaneously with the HMW formulation compared to around 45% with the control gel formulation. A 1% HMW chitosan formulation can be used to enhance the transdermal penetration of retinoids to maximize the therapeutic power of retinoids.

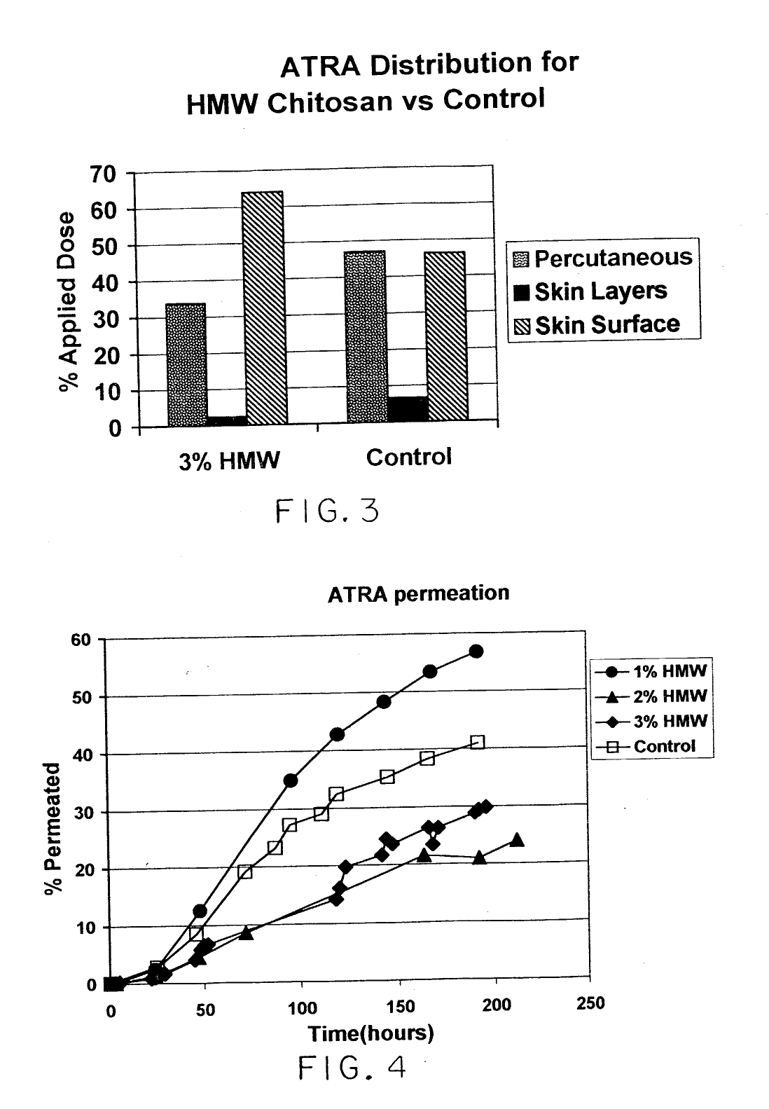

FIG. 3 shows that the 3 % HMW chitosan [containing 0.1% ATRA (0.1 g ATRA, 0.04 g butylated hydroxytoluene (BHT), 1 g of Cremophor® RH40, 15 g ethanol (200 proof), 3 g of Chitosan HMW TD012, 80.8 g water, 1 g of glacial acetic acid] compared to a standard control gel [containing the following: 0.1 g ATRA, 0.04 g BHT, 1 g of Cremophor® RH40, 15 g of ethanol, 0.5 g of Carbopol 940 NF, 76 g water and 0.7 g Triethanolaminel. A lower percutaneous penetration was obtained with the 3% HMW compared with the control gel. 32% of the applied retinoid dose was delivered percutaneously with the HMW formulation compared to 45% with the control gel formulation. A 3% HMW chitosan formulation could be used to control release the retinoids and limit the potential for irritation.

FIG. 4 shows the ability to release ATRA from the chitosan formulations is highly dependent on their viscosity which range from 552 cps for 1% HMW to 171,163 cps for the 3% HMW estimated from the Philipof's equation: V=(1+KC)⁸, where V is the viscosity in cps, K is a constant, C is the concentration expressed as a fraction. The higher the viscosity of the HMW, the slower the percutaneous release of ATRA over a period of 220 hours of a single application in a Franz cell apparatus. The topical control gel consisting of Carbopol® 940 NF polymer displays a percutaneous ATRA delivery which lies somewhere in between the Topical ATRA formulations ranging from 1% to 3% HMW.

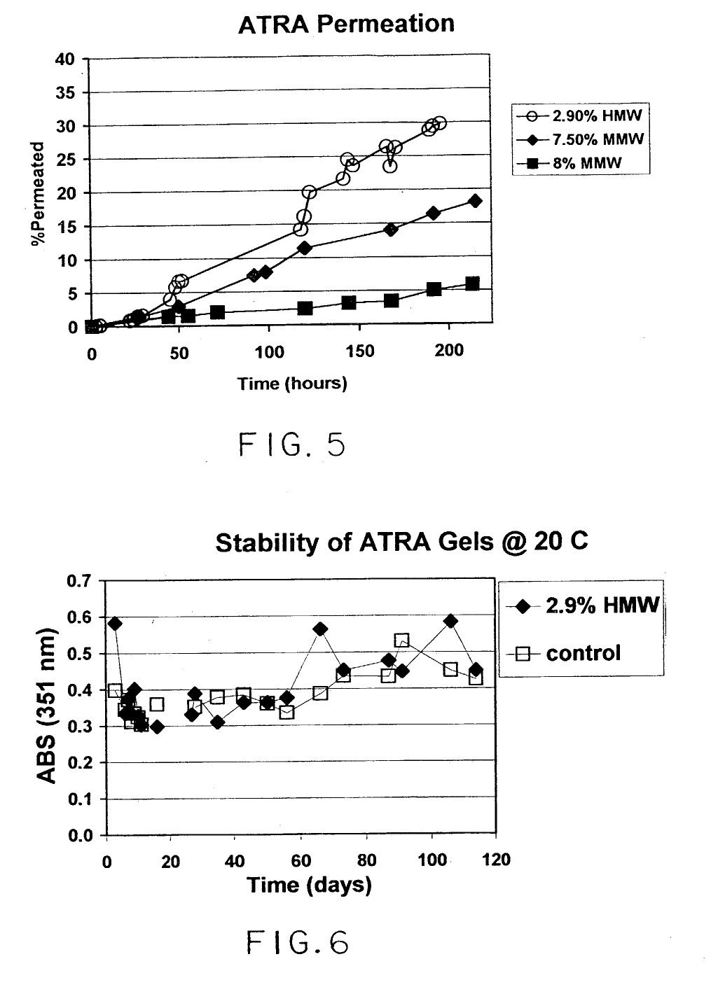

In FIG. 5, the percutaneous permeation of MMW chitosan gels of high viscosity (viscosity of 3.27 million cps for the 8% MMW estimated from the Philipof's equation) compared to a 2.9% HMW with an estimated viscosity of 117,163 cps). The topical ATRA formulations containing the higher viscosity chitosan display a lower percutaneous penetration through hairless mouse skin after 220 hours of continuous application in a Franz cell apparatus.

One of ordinary skill in the art can readily select an appropriate chitosan component as the carrier for the compositions and methods of the present invention, based upon the teachings described herein. For example, as described above, one of ordinary skill in the art can use Phillipof's equation for predicting release rates from polymer concentrations and viscosities. As aforesaid, a lower viscosity chitosan used at higher concentrations will provide similar release rates as a higher viscosity chitosan. Thus, if it is desirable to have a slow release of the retinoids, one would select a carrier base having a high viscosity chitosan, e.g., a chitosan with molecular weight of at least about 100,000 Dalton, e.g., 300,000, at a concentration of least 2 weight %. This type of composition is desirable to minimize the overload of retinoids which may lead to irritation of the skin.

Alternatively, if it is desirable to have a faster release of the retinoid, one would select a chitosan solution having a high molecular weight, e.g., of at least about 250,000, at a lower concentration, e.g., from about 1% to about 2%. Such compositions are useful for increasing the transdermal release of the active agent over a shorter period of time.

The combination of chitosan and retinoids in the compositions of the present invention enhances the normal tissue architecture of naturally and photoaged skin while reducing skin irritation, normally seen with retinoid preparations.

The compositions of the present invention can be formulated into gels, lotions, ointments or creams according to known methods. The delivery systems can be used to form gels at concentrations greater than 2%. In addition, these gels can be used as is or formed into creams by including an oil and emulsifying the mixture, by known methods. Preferred oils include avocado oil, sea buckthorn oil, jojoba oil, etc. Other compounds can also be added as desired to increase the effectiveness of the formulations. Examples of such additives may include, but are not limited to, vitamins such as A, B, C, D, E, K, etc., moisturizers such as alpha-hydroxy acids, etc. Other additives may be used to improve the appearance of the formulation, e.g., odor, texture or visual appeal. Examples of such additives include, but are not limited to, fragrances, coloring, emollients and ingredients for the enhanced percutaneous absorption of various therapeutic actives, such as glycerol, propylene glycol, oleic acid, surfactants, etc.

The delivery systems of the present invention can contain a large number of pharmaceutical and therapeutic actives that can be applied topically either singularly or in combination. Examples of these actives include, but are not limited to compounds such as the following: Anti-fungal agents such as Imidazoles, Clotrimazole, Clotrimazole/betamethasone dipropionate, Econazole, Ketoconazole, Miconazole, Oxiconazole, Sulconazole, Allylamines, Naftifine, Terbinafine, Polyenes, Nystatin, Nystatin/triamcinolone, Ciclopirox olamine, Triacetin/sodium propionate/benzalkonium chloride/chloroxylenol, Tolfanate, Undecylenic acid/zinc, undecylenate. Anti-inflammatory agents such as coal tar, shale tar, wood tar, non-steroidal anti-inflammatory drugs (NSAIDS) salicylic acid, salicylate esters and salts, acetylsalicylic acid, and the like. Local anaesthetics such as cocaine, benzocaine, tetracaine, lidocaine, bupivacaine, their hydrochloride salts, and the like. Antibiotic agents such as bacitracin, mupirocim, erythromycin, neomycin, clindamycin, doxycycline, trimethoprim-sulfamethoxazole, penicillin-V, trimthoprim-sulfamethoxazole, chloramphenicol, gentamycin, azithromycin, ciprofloxacin, ofloxacin, ceftriaxone, minocycline, amoxicillin-clavulanate, first-generation cephalosporin, ceftriaxone, and the like. Sulfanilamide antibacterial agents such as sulfanilamide, sulfacetamide, sulfadiazine, sulfisoxazole, sulfamethoxazole, trimethoprim, pyrimethamine, and the like. Antiviral agents such as Imiquamod, acyclovir, valacyclovir, famcyclovir, penciclovir, idoxuridine, trifluridine, foscarnet, cidofovir, interferons, IFN-α, IFN-α2b, IFN-αn3, nucleoside analogues, protease inhibitors and the like. Antiseptic agents such as acridine dyes, alcohols, bronopol, chlorhexidine, phenols, hexachlorophene, organic mercurials, organic peroxides, i.e., benzoyl peroxide, quaternary ammonium compounds, and the like. Vitamin and vitamin derivatives such as Vitamin A, retinol, retinoic acid (both cis and trans), alpha-tocopherol (Vitamin E), 7-dehydrocholesterol (Vitamin D), Vitamin K, thiamine riboflavin, niacin, pyridoxine, biotin, pantothenic acid, ascorbic acid, choline, inositol, and the like. Anti-inflammatory corticosteroids such as progesterone, hydrocortisone, prednisone, fludrocortisone, triamcinolone, dexamethasone, betamethasone, fluocinolone, and the like. Autacoids such as prostaglandins, prostacyclin, thromboxanes, leukotrienes, angiotensins (captopril), as well as other pharmaceutically active peptides such as serotonin, endorphins, vasopressin, oxytocin, and the like. Kerolytic agents such as benzoyl peroxide, salicylic acid, trichloroacetic acid, and piroctone, and wart treatment compounds such as salicyclic acid, trichloroacetic acid and lactic acid, singularly or in combination with anti-viral agents. Anti-alopecia agents such as niacin, nicotinate esters and salts, and minoxidil. Sun-Protective agents such aminobenzoates, Para-aminobenzoic acid (PABA), Ethyl-4-[bis(hydroxypropyl)-aminobenzoate, Glyceyl PABA, Amyl p-dimethylaminobenzoate (padimate A), 2-ethylhexyl PABA (padimate O), Cinnamates, Dietholamine p-methoxycinnamate (Parsol MCX), Salicylates, 2-ethylhexyl salicylate, Homosalate (homomenthyl salicylate), Octyl salicylate, Triethanolamine salicylate, Trolamine salicylate, Benzophenones, Dioxybenzone, Sulisobenzone, Oxybenzone, Ethylhexyl, 2-cyano-3,3-diphenyl-acrylate (octocrylene), Lawsone and dihydroxyacetone, 2-phenylbenzimidazole-5-sulfonic acid, Digalloyl trioleate, Red veterinary petrolatum, Titatium dioxide, Methyl anthranilate, Butylmethoxydibenzoyl methane (avobenzone), zinc oxide.

Other additives can also be used, e.g., moisturizing agents such as lactic acid, pyrrolidone carboxylic acid, glycolic acid, water, glycerine, propylene glycol, sorbitol, other alphahydroxy carboxylic acids, and various salts of these esters and salts, and the like and additives for the enhanced percutaneous absorption of various pharmaceutical or therapeutic actives. Such percutaneous enhancers include propylene glycol, glycerol, urea, diethyl sebecate, sodium lauryl sulfate, sodium laureth sulfate, sorbitan ethoxylates, nicotinate esters (such as hexyl nicotinate), oleic acid, pyrrolidone carboxylate esters, (such as dodecyl pyrrolidone carboxylate), N-methyl pyrrolidone, N,N-diethyl-mtoluamide, dimethyl sulfoxide, decyl methyl sulfoxide, alkyl methyl sulfoxides, N,N-dimethyl formamide, cis-11-octadecenoic acid, 1-dodecylazacycloheptan-2-one, and 1,3-dioxacyclopentane or 1,2-dioxacyclohexane containing at least one aliphatic group of four to eighteen carbon atoms.

The amount of active employed will be that amount necessary to deliver a pharmaceutically or therapeutically effective amount to achieve the desired result at the site of application. In practice, this will vary depending upon the particular medicament, severity of the condition as well as other factors. In general, the concentration of the actives in the delivery systems can vary from as little as 0.0001 up to 5 percent or higher, by weight of the delivery system. For retinoids, a preferred dose is between 0.01%-1% for retinol and between 0.01%-0.1% for all-trans-retinoic acid.

Other adjuvant ingredients such as glycerin, propylene glycol, sorbitol, preservatives, stearic acid, cetyl alcohol, other high molecular weight alcohols, surfactants, menthol, eucalyptus oil, other essential oils, fragrances, penetration enhancers, and the like to give stable cremes, ointments, lotions, aerosols, solutions, may also be included.

Alternatively, solutions or mixtures of the actives with the chitosan derivatives may be prepared with or without some of the adjuvant ingredients, and these solutions or mixtures may be fabricated into films, rods, sheets, sponges or fibers for use as suppositories, medicated sutures, medicated sheets, medicated bandages, patches, and the like. It is relatively easy to process chitosan into various forms such as small particles, gel, and cotton mesh for drug delivery applications. Such methods are known in the art.

In a preferred composition, alpha-hydroxy acid (AHA) is used to completely dissolve the chitosan. AHA is also referred to as glycolic acid in the methods and examples described below. The benefit of using alpha-hydroxy acid is two-fold. One advantage is that it helps dissolve the chitosan. Another advantage is that the combination of alpha-hydroxy acid and chitosan, which is basic, raises the pH of the composition which in turn, minimizes the peeling seen with standard alpha-hydroxy acid formulations. Neutral or mildly acidic vehicles of alpha-hydroxy acids are actively being sought (Neudecker et al., 2000). It is conmmon practice to use ammonium salts to neutralize the alpha-hydroxy acids present in most current cosmetic preparations. Ammonium salts present in most current cosmetic preparations of alpha-hydroxy acids may prevent hyaluronan (HA) enhancement (Neudecker et al., 2000). Chitosan, through the presence of its amino groups on the polymer chain, can be used to neutralize the alpha hydroxy acids. The addition of 3% HMW chitosan raises the pH of an alpha hydroxy solution from 3.5 to 5.5 thus bringing the pH of the AHA formulation in the mildly acidic range where the action of AHA can effect the ability to stimulate HA production rather than implement their action by peeling the skin and cause diffuse wound healing.

AHA is thus useful as an active agent alone, or in conjunction with another pharmaceutical or therapeutic active.

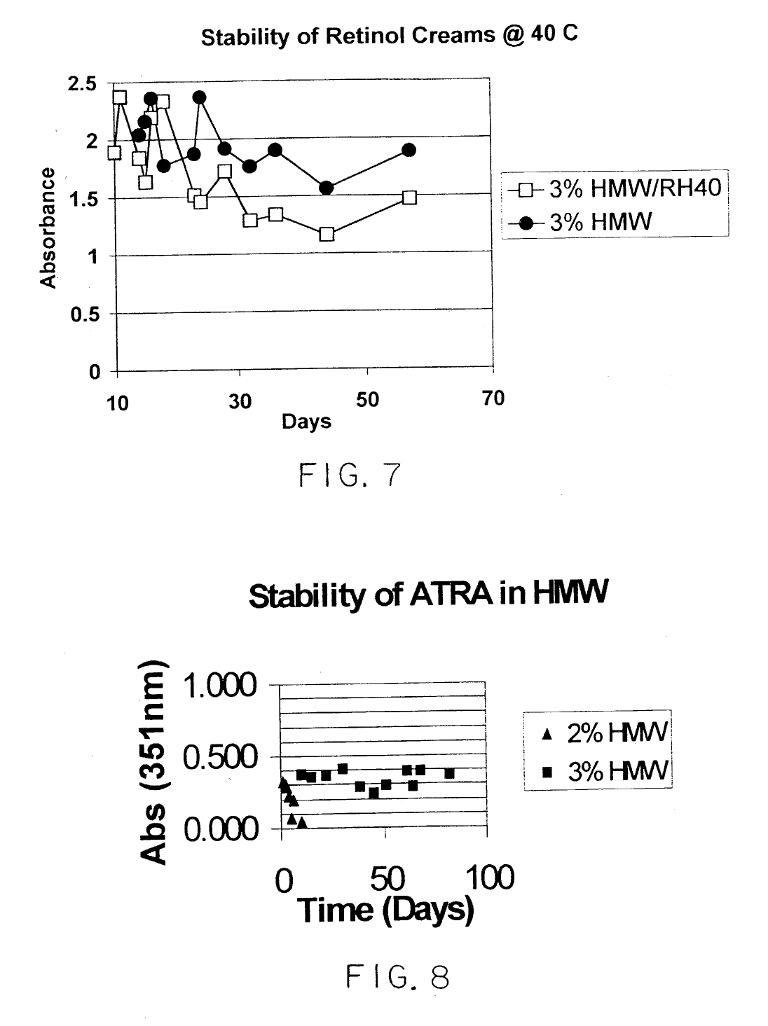

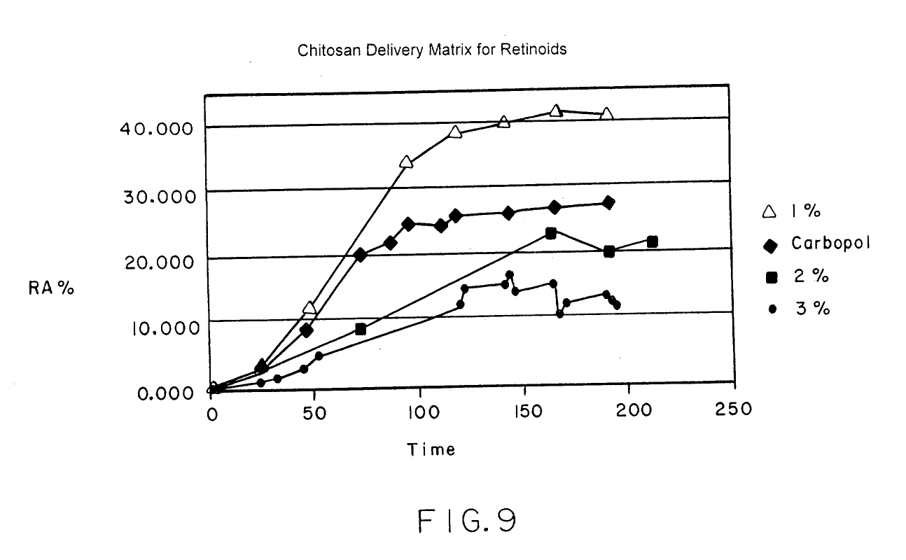

The compositions of the present invention are stable, as is necessary for topical treatments. ATRA gels made from the HMW chitosan at concentrations greater than 2% are stable for at least 120 days and comparable in stability to the standard control gels made from Carbopol as shown in FIG. 6. Lower concentrations of chitosan may cause a reduction in the stability of the ATRA in the gel formulation. As shown in FIG. 7, creams made from the 3% HMW are highly stable, again as a result if the high viscosity of this type of chitosan when present at greater than 2% concentration. Similar results would be obtained with the MMW chitosan present at concentration than 5% w/w. The difference in stability is related to the addition of the surfactant Cremophor RH40 which causes a reduction in ATRA stability compared to the HMW formulation alone.

The inventors have found that the use of a carrier base with a high-viscosity grade chitosan, e.g., having a molecular weight of at least about 300,000 Dalton and at a concentration, e.g., of at least 2 weight % results in a greater stability of the retinoid preparation, over a period of months. See FIG. 8 and Example 3, below. Thus, one advantage of using a high molecular weight chitosan in delivering an active agent, such as retinoids, is the ability to use a lower concentration to obtain a sufficient viscosity required for stabilization of the retinoids. Stability of formulations is often tested at 40° C. for a period of several months.

To the best of our knowledge there are presently no chitosan-based retinoid delivery systems. For percutaneous drug delivery chitosan offers unique advantages. For example, chitosan is used in cosmetology to make moisturizing creams. The concentration in moisturizers and soaps varies from 0.3% to 1% chitosan. These concentrations have been experimentally tested by the manufacturers and are well tolerated on the skin. It is also used in hair sprays, styling gels and shampoos: its cationic nature enables a close bond to the keratin anion (Sachetto, 1986; Cleenewerck, 1994). Chitosan is a biodegradable polymer which has advantages over a synthetic polymer, e.g., PP2. For example, chitosan is completely degraded in the body. It degrades without leaving residual matter which could build up in the tissues. As suture material, chitosan has been shown to be completely absorbed in one to two months so it would release the drug during the same period (Suzuki, 1995). It is unnecessary to remove chitosan from the body after the complete release of the drug because chitosan has good biodegradability and is completely dissolved by enzymes such as lysozyme.

As aforesaid, the present invention provides methods for the treatment of many skin ailments. To our knowledge there is no controlled topical delivery system of retinoids for melanoma chemoprevention. One aspect of the present invention is a chitosan based percutaneous delivery system for the chemoprevention of melanoma in individuals with dysplastic nevi who are at high risk of developing melanoma.

In addition, the combination of retinoids and a chitosan-based delivery system takes advantage of the immunostimulating properties of chitosan for the delivery of therapeutic actives in skin conditions that necessitate an immune response. The compositions of the present invention utilize the property of chitosan to initiate immune and reparative functions, either directly or indirectly through the stimulation of macrophages in the skin tissue.

Activation and production of cytokines such as IL-1 leads to increased angiogenesis and skin reparative functions. IL-1 and TNF-α, produced by macrophages, stimulate fibroblasts (Chang J et al. 1986). Chitosan has been shown to stimulate macrophage production, resulting in activation of cytokines such as interleuken-1 (IL-1) and interferon gamma (IFN-γ). (Chensue et al., 1989; Shibata et al., 1997). The degree of deacetylation for immunostimulatory activity is optimal around 70% and other degrees of deacetylation result in the reduction of immunostimulatory activity (Nishimura et al, 1984, 1985, 1986, 1990). A 70% deacetylated chitin has been used in combination with petrolatum to immunostimulate the skin in the management of senile erythroderma. (Horuchi & Otoyama, 1996). The chitin derivative is not employed in these studies as a delivery system but rather as the active ingredient in the topical petrolatum-based formulation.

In addition, the chito-oligomers released from chitosan by the in vivo hydrolytic action of lysozyme and N-acetyl-β-D-glucosaminidase after penetration of chitosan into the skin may stimulate hyaluronan synthesis. Recent evidence is found for the presence of DG42 protein (a chito-oligomer synthase) during embryogenesis, producing chito-oligomers acting as primers in the synthesis of hyaluronan. Overexpression of DG42 in mouse cells leads to the synthesis of chito-oligomers, and hyaluronan synthase preparations also contain chitin synthase activities (Varki A, 1996; Semino et al., 1996; Bakkers et al., 1997).

Chitosan has the potential, directly or indirectly through the formation of hyaluronic acid, to correct this deficiency and to provide correct deposition of collagen fibers such as reduced space and fiber thinness, fiber disorganization and depth of disorganization.

Therefore the administration of retinoids via a chitosan carrier base has the potential of enhancing both the quantity and quality of new collagen production in skin connective tissue.

The methods of the present invention take advantage of the reparatory effect of chitosan to stimulate fibroblasts in conjunction with the therapeutic effect of retinoids to obtain a synergistic effect. The increase in collagen repair is useful for treating conditions that which would benefit from an irnmunostimulatory response, e.g., in preparations used for anti-wrinkle products as well as for products that are used to treat photodamage and other such skin conditions.

As aforesaid, the compositions of the present ivention are useful for treating skin diseases. Examples of skin diseases which can be treated include, but are not limited to, acne, melanoma, premature skin aging, and photodamage. In preferred embodiments the active agent comprises a pharmaceutical active, e.g., an agent that is used for the treatment of skin diseases. Examples of pharmaceutical actives include, but are not limited to retinoids, such as corticosteroids, non-steroidal anti-inflammatory drugs (NSAIDS), hormones, anti-fungal agents, anti-septic agents, local anaesthetics, kerolytic agents, 5-FU, etc. In other embodiments, the active agent comprises a therapeutic active, e.g., vitamins, moisturizing agents such as alpha-hydroxy acids, etc., as further described below. The amount and frequency of the application of the delivery systems can readily be determined by one of ordinary skill in the art, based upon the type and severity of the ailment, as well as the amount of agent present in the system.

As aforesaid, in some methods of treating certain skin diseases, it may be useful to use the compositions of the present invention in conjunction with other treatments for the disease. For example, in treating precancerous skin conditions, it may be useful to use the compositions of the present invention with standard treatments that use an anti-cancer drug, e.g., 5-FU, for the treatment of actinic keratosis.

The present invention is further illustrated by the following Examples. The Examples are provided to aid in the understanding of the invention and are not construed as a limitation thereof.

All examples are carried out using standard techniques, which are well known and routine to those of skill in the art, except where otherwise described in detail. Routine techniques of the following examples can be carried out as described in standard laboratory manuals.

### EXAMPLES

Summary of Experiments:

In the design of the topical delivery system different polymer formulations were prepared. Table 2 shows the types of chitosan used. The chitosan was obtained from Primex Ingredients, Avaldnes, Norway.

These formulations were then tested in in vitro assays, i.e. penetration and recovery studies using conventional and radiolabeled retinoids and long-term stability studies at 20° C. and 40° C., as described below with a Franz diffusion cell. Human subjects are then exposed to selected formulations (in vivo) and compared to current dermal retinoid formulation to test their ability to reduce irritation.

TABLE 2

| TYPE OF | VISCOS- | DEGREE OF | |
|---|---|---|---|
| CHITOSAN | ITY¹ | DEACETYL- | |
| (LOT #) | (MPAS) | ATION² | DESCRIPTION |
| HMW | 552 | 89.0% | Soluble in 1% Acetic Acid |
| (TD012) | | | or 2% Glycolic Acid |
| | | | Gel at concentration of |
| | | | 3% |
| MMW | 66 | 96.1% | Soluble in 1% Acetic Acid |
| (TM761) | | | or 2% Glycolic Acid |
| | | | Gel at concentration of |
| | | | 5% or higher |
| LMW | 7 | 95.0% | Soluble in 1% Acetic Acid |
| (TM615) | | | or 2% Glycolic Acid |
| | | | Slightly viscous liquid at |
| | | | concentration of 3% |
| LMW | 23 | 80.8% | Soluble in 1% Acetic Acid |
| (TM816) | | | or 2% Glycolic Acid |
| | | | Slightly viscous liquid at |
| | | | concentration of 3% |
| LMW | 10 | 87.8% | Soluble in 1% Acetic Acid |
| (TM611) | | | or 2% Glycolic Acid |
| | | | Slightly viscous liquid at |
| | | | concentration of 3% |

¹The viscosity of 1% solutions in 1% acetic acid was measured on a Brookfield LVT viscometer, 25° C., with appropriate spindle at 30 rpm (From Primex Ingredients, Product Literature).
²The degree of deacetylation was measured by the UV-method (From Primex Ingredients, Product Literature).

In the following examples, sample TD012 is an example of a high molecular weight (HMW) chitosan, TM761 is an example of a middle molecular weight (MMW) chitosan, and TM615, TM816 and TM611 are examples of low molecular weight (LMW) chitosans.

### Example 1

Preparation of Chitosan-Retinoid Compositions

Gel Chitosan TD012 has a viscosity of 500 cP when dissolved with 1% glacial acetic acid at 1% concentration. The viscosity increases as a function of concentration of the polymer, reaching an estimated 171,163 cps at 3% concentration.

Colloidal solutions up to 3% (wt/wt) chitosan were obtained by dissolving high molecular weight chitosan (HMW (TD012); MW 360,000 Daltons) in 1% glacial acetic acid at room temperature. Carrier bases up to 8% were obtained by suspending chitosan powder of middle molecular weight (MMW (TM761); MW 120,000) (8 g in 66 g of deionized water) in water at room temperature, raising the temperature to 90° C. and adding 25 g of water and 1 g of glacial acetic acid, dropwise to chitosan to form a clear, highly viscous solution after cooling at room temperature.

### Example 2

In Vitro Skin Penetration Studies Using Radiolabeled Retinoids.

Fresh hairless mouse skin samples were obtained from surgery, and upon arrival to the lab they were stored in a freezer (−20° C.). Immediately prior to the permeation experiments, skin samples without subcutaneous fat were thawed by floating on water at 22° C. for about 10-20 minutes. A 1.0 cm² portion of the skin samples was fastened between the Franz diffusion cell's receptor chamber and chimney top by an o-ring and a spring clamp (PermeGear, Inc.) (Lehman et al., 1988.)

For gel sample preparation, 20 uL of radiolabeled ³H-Retinoic Acid (20 microcuries) (NEN LifeSciences, Boston, Mass.) were added to 1.5 grams of a retinoic acid stock solution, comprised of 100 mg of retinoic acid in 15 grams of absolute ethanol (200 proof) and 1 g of hydrogenated castor oil (cremophor RH40, BASF Corporation) and were mixed with 8.5 grams of the chitosan colloidal solution.

For the cream sample preparation, 20 uL of radiolabeled ³H-Retinoic Acid (20 microcuries) (NEN LifeSciences, Boston, Mass.) were added to 0.6 g of the retinoic acid stock solution (comprised of 100 mg of retinoic acid, 10 g of avocado oil and 1 g of Cremophor RH40). The solution was then mixed with 1.5 g of glycerin, 0.05 g of Vitamin E, 0.1 g of Seabuckthorn Seed Extract.

Finally, 7.8 g of TD012 (2.9%) chitosan, dissolved in glycolic acid (pH 5.5) was added homogeneously.

Approximately 200 mg of each formulation was applied to the sample compartment (i.e. the epidermal side) of the skin sample. The dermal surface of the skin was perfused with receptor phase solution (phosphate buffered saline containing 0.5% Volpo surfactant (Croda, Inc.). Each formulation was tested in triplicate.

The receptor volume was sampled every 24 hours by withdrawing 500 gL. It was then mixed with scintillation fluid for scintillation counting.

At the end of the run the entire content of the reservoir compartment of the Franz cell (5 ml) was removed and placed in a scintillation vial with 10 ml of scintillation fluid. Any retinoid remaining on the surface of the skin (top wash) was extracted with 2×500 μL of ethanol containing 1% glacial acetic acid and placed in a scintillation vial containing 9 ml of scintillation fluid (Packard).

The epidermis and dermis were digested overnight in 4 ml of tissue solubilizer (Solvable Tissue and Gel Solubilizer-Packard Instruments) to which 6 ml of scintillation fluid (Ultima Gold-Packard Instruments) was added and analyzed by scintillation counting.

The permeation of all-trans retinoic acid (ATRA) across hairless mouse skin as a function of concentration of the high viscosity chitosan TD012 and middle viscosity TM761 is shown in FIG. 1. As shown in FIG. 1, it is possible to increase the percutaneous penetration from 8% to 68% ATRA percutaneous penetration by changing the chitosan polymer from 8% TM761 (the medium viscosity chitosan: 10 cP at 1% concentration) to 1% TD012 (high viscosity chiosan: 552 cP at 1% concentration). As the amount of ATRA penetrating increases, there is a concomitant decrease of ATRA on the skin surface. The amount in the skin layers decreases from 5% to 0.5% as the amount of ATRA penetrated decreases.

As the concentration of the high viscosity chitosan (TD012) decreases, the amount of ATRA permeated through the skin into the Franz Cell Reservoir compartment increases as shown in FIG. 4. The ATRA release from a standard gel made with Carbopol™ 940 NF acrylate polymer (BF Goodrich) is intermediary between the 1% and the 2% chitosan TD012.

These results show that it is possible to control the delivery of the retinoid ATRA by changing the chitosan concentration, in relation to the viscosity of the chitosan. An increase in concentration of the middle viscosity chitosan TM761 further reduces the permeation rate (FIG. 5).

### Example 3

Stability Testing of Retinoid Gels and Creams

A. Preparation of Gels and Creams Based on Retinoic Acid and Chitosan TD012.

For the preparation of gels and creams the high molecular weight TD012 chitosan (M.Wt 360,000 Dalton) was chosen due its slow release characteristics for retinoic acid. We chose to use the TD012 Chitosan (2.9%) because it forms a highly viscous colloidal solution at room temperature and it offers a favourable ATRA release profile.

Preparation of Retinoic Acid Gel.

Solution A was prepared by dissolving chitosan in a 1% glacial acetic acid solution as follows: 2.9% Chitosan TD012, 79.98% Water in 1% Acetic Acid. Solution B was prepared by dissolving cremophor RH40 in ethanol in an amber container followed by BHT and retinoic acid. The amounts are as follows: 15% Ethanol, 1% Cremophor RH40, 0.02% BHT and 0.1% Retinoic Acid. Solution B was mixed into solution A using a 3-blade laboratory mixer.

Preparation of Retinol Cream was as Follows:

| | | |
|---|---|---|
| Solution A: | 3% | Chitosan TD012 |
| appx. | 62.84% | Water |
| | 2.86% | Glycolic Acid (70% solution) |
| appx. | 3.5% | NaOH Solution (10 g in 100 ml water) |
| | | to bring to pH = 3.5 |
| Solution B: | 15% | Glycerin |
| | 1% | Cremophor RH40 |
| | 0.5% | Vitamin E Acetate |
| | 10% | Avocado Oil |
| | 1% | Sea buckthorn Seed Oil |
| | 0.1% | Perfume |
| | 0.2% | Retinol 50C |

Solution A was prepared by adding glycolic acid to water. While stirring, NaOH (10 g/100 ml) was added dropwise to raise the pH from 2.12 to 3.5. Then chitosan was added and allowed to dissolve completely overnight. The final pH was 5.3-5.5. Solution B was prepared by combining the glycerin, cremophor RH40, vitamin E acetate, avocado oil, and sea buckthorn oil. The perfume and retinol SOC (50% w/w of retinol in Polysorbate 20-BASF) were added sequentially to obtain an homogeneous solution. Solution B was then incorporated into Solution A using a 3-blade laboratory stirrer.

Preparation of 0.1% Retinoic Acid Cream

For the retinoic acid cream 100 mg retinoic acid was substituted for the 200 mg of retinol 50 C. The retinoic acid was initially suspended in 10 g of avocado oil containing 1 g of cremophor RH40. The rest of procedure is similar to the retinol cream.

Stability Testing

The stability of the retinoic acid gels was tested at both 20° C. and 40° C. in a water bath. Retinoid concentrations were tested by dissolving 0.2 g of the gel (or cream) in 6.7 g of a 1% acetic acid in ethanol solution. The solution was then stirred using a magnetic stirring bar and plate until the retinoid and chitosan had dissolved.

For the retinoic acid sample, a 100 μL quantity was diluted 10-fold in 1% acetic acid/ethanol solution and the absorbance measured at 351 nm using a Pharmacia Biotech Ultrospec 2000 Spectrophotometer. For the retinol samples, a 50 μL quantity was diluted 20-fold in 1% acetic acid/ethanol solution and absorbance readings at 326 nm. The stability measurement was repeated once per week over several weeks.

Gel samples designated 87-1 consist of 0.1% ATRA in 2.9% TD012 as in EXAMPLE 1; 101-1 is 0.1 % ATRA in 0.5% Carbopol 940 NF instead of 2.9 TD012; 109-1 is 0.1% ATRA as in EXAMPLE 1 with 3.5% TD012 instead of 2.9% TD012. Cream samples 2-3-1 consist of 1% retinol in TD012 (2.9%) as in EXAMPLE 2. Cream sample 2-5-1 is the same as 2-3-1 without the Cremophor component.

### Example 4

Patch Testing in Healthy Individuals

Human studies are undertaken to evaluate the irritation potential of the chitosan/ATRA percutaneous delivery system. 15 patients having signed an informed consent are patch tested with commercial creams containing conventional ATRA and with a cream of the present invention containing chitosan and retinoids at an equivalent dose. The creams are prepared according to the methods in Example 3 and as shown below. The irritant potential of the tretinoin/chitosan delivery system on human skin is assessed by means of patch test evaluations as follows:

For assessing irritation (Seaton, 1995), the occlusive Hill Top Chamber patch testing system (Hill Top Research, Inc., Cincinnati, Ohio) incorporates 0.2 ml of sample.

The human evaluation involved three strengths of commercially available tretinoin (ATRA) cream (0.01%, 0.05% and 0.1%) with two concentrations of chitosan (1% and 3%) in the formulation.

The data is evaluated in terms of a Mean Irritation Score by evaluating the extent of erythema, as previously described (Mills and Berger, 1998). Statistical evaluation includes both frequency and severity of erythema seen at sites treated with tretinoin containing chitosan and commercially available tretinoin using analysis of variance (ANOVA) and the paired t-test.

Patch Testing of ATRA Cream

The drug product (ATRA Cream) consists of a modified retinoic acid formulation. The control cream was obtained from Technical bulletin ME 142e for Retinoic acid (BASF Corporation, N.J.). To test the chitosan-based cream on irritancy levels the following formulations are prepared:

| Control Cream | | | |
|---|---|---|---|
| I | Luvitol ® EHO (1) | 8 | g |
| II | Cremophor A 6 (1) | 3.0 | g |
| | Cremophor A 25 (1) | 1.5 | g |
| | Glycerol monostearate | 3.0 | g |
| | Cetyl alcohol | 3.0 | g |
| | Tegiloxan ® 100 (2) | 0.5 | g |
| III | Butylated hydroxytoluene | 0.04 | g |
| | 1,2-Propylene glycol | 4.0 | g |
| | Nip-Nip ® (3) | 0.2 | g |
| | Germail ® (4) | 0.3 | g |
| | Perfume | 0.2 | g |
| | Water | 76.2 | g |

Mixture II is heated to 75° C. and stir in Solution I. Mixture III is heated until a completely clear solution is obtained, then added to the heated Mixture I/II and stirred until cold.

| Control Cream + 0.1% ATRA | | | |
|---|---|---|---|
| I | ATRA (USP) | 100 | mg |
| | Luvitol ® EHO (1) | 8 | g |
| II | Cremophor A 6 (1) | 3.0 | g |
| | Cremophor A 25 (1) | 1.5 | g |
| | Glycerol monostearate | 3.0 | g |
| | Cetyl alcohol | 3.0 | g |
| | Tegiloxan ® 100 (2) | 0.5 | g |
| III | Butylated hydroxytoluene | 0.04 | g |
| | 1,2-Propylene glycol | 4.0 | g |
| | Nip-Nip ® (3) | 0.2 | g |
| | Germail ® (4) | 0.3 | g |
| | Perfume | 0.2 | g |
| | Water | 76.2 | g |

Mixture II is heated to 75° C. and stir in Solution I. Mixture III is heated until a completely clear solution is obtained, then added to the heated Mixture I/lI and stirred until cold.

| 3% HMW-Chitosan Cream | | | |
|---|---|---|---|
| I | Glycerol | 15 | g |
| | Cremophor ® RH40 (1) | 1 | g |
| | Vitamin E Acetate | 0.5 | g |
| | Avocado Oil | 10 | g |
| | Sea Buckthorn Seed Oil | 1 | g |
| | Perfume | 0.1 | g |
| II | Chitosan TD012 | 3.0 | g |
| | Glycolic Acid (70%) | 2.86 | g |
| | NaOH Solution (10%) | 3.5 | g |
| | Water | 62.84 | g |

Mixture I is incorporated with solution II and the Mixture I/II is homogenized to a fine consistency.

| 3% HMW-Chitosan Cream + 0.1% ATRA | | | |
|---|---|---|---|
| I | ATRA | 100 | mg |
| | Glycerol | 15 | g |
| | Cremophor ® RH40 (1) | 1 | g |
| | Vitamin E Acetate | 0.5 | g |
| | Avocado Oil | 10 | g |
| | Sea Buckthorn Seed Oil | 1 | g |
| | Perfume | 0.1 | g |
| II | Chitosan TD012 | 3.0 | g |
| | Glycolic Acid (70%) | 2.86 | g |
| | NaOH Solution (10%) | 3.5 | g |
| | Water | 62.84 | g |

Mixture I is incorporated with solution II and the Mixture I/Il is homogenized to a fine consistency.

Product Suppliers and Manufacturers

1. BASF Corporation, Ludwigshafen, Germany

2. Th. Goldschmidt AG, Essen, Germany

3. Henkel KgaA, Dusseldorf, Germany

4. Ru-Jac Inc., Upper Montclair, N.J.

Clinical Experimental Design—The Clinical Study is Performed in Three Parts: Part I

Part I involves 6 human volunteers. Each volunteer receives the 6 formulations listed below. Each formulation consists of 0.2 g of test sample, applied to the volar forearm (3 formulations on each forearm) in the form of a patch (Hill Top Research, Inc., Cincinnati, Ohio). Each human subject is evaluated at 24 hours for signs of irritancy (e.g. erythema).

| Patients No. 1 to 6: | Formulation (A, B, C, D as referred above) |
|---|---|
| Site 1 | A (Control Cream) |
| Site 2 | B (Control Cream + 0.1% ATRA) |
| Site 3 | C (1% HMW-Chitosan) |
| Site 4 | D (1% HMW-Chitosan 1% + 0.1% ATRA) |
| Site 5 | C (3% HMW-Chitosan) |
| Site 6 | D (3% HMW-Chitosan) +0.1% ATRA) |

The location of each test sample is rotated for each individual according to latin square design.

Part II

Given that the results of Part I show no irritation from the volar application of the formulations, Part II involves 3 additional human subjects, each subject receiving 3 patches containing 0.2 grams of test sample to the paraspinal area of the back to verify any irritation caused by the base alone without ATRA. The patch application is for 24 hours with irritancy evaluation at 30 minutes after patch removal and 24 hours after patch removal.

| For Patients 7 to 9 | Formulation (A, C as referred above) |
|---|---|
| Site 1 | A (Base Cream) |
| Site 2 | C (HMW-Chitosan 1%) |
| Site 3 | C (HMW-Chitosan 3%) |

The location of each test sample is rotated for each individual according to latin square design.

Part III

Given that the results of Part II show no irritancy, Part III involves the testing of 6 additional human subjects. Each participant receives 6 patches applied to the paraspinal area on the back, including 3 patches of the control cream and 3 patches of the 3.9% HMW-chitosan cream each containing 3 strengths of ATRA. Patches are removed after 24 hours and irritancy scored 30 minutes and 24 hours. Statistical evaluation includes ANOVA and paired t-test to evaluate any significant difference between treatments, sites and patients.

| For Patients 10 to 15 | Formulations (B, C as referred above) |
|---|---|
| Site 1 | B (Control Cream + 0.01% ATRA) |
| Site 2 | B (Control Cream + 0.05% ATRA) |
| Site 3 | B (Control Cream + 0.1% ATRA) |
| Site 4 | D (3% HMW-Chitosan + 0.01% ATRA) |
| Site 5 | D (3% HMW-Chitosan + 0.05% ATRA) |
| Site 6 | D (3% HMW-Chitosan + 0.15% ATRA) |

The location of each test sample is rotated for each individual according to latin square design.

### Example 5

Chitosan Gels as Delivery Vehicles for Retinoic Acid

The topical carrier base consisting of high viscosity chitosan with a molecular weight of at least 300,000 Dalton and at a concentration of at least 2 weight % acts as a delivery system to control the release of retinoic acid (RA). Studies with [3H]retinoic acid. A high molecular weight chitosan (viscosity of 552 cP with 1% solutions in 1% acetic acid measured on a Brookfield LVT viscometer at 25 C., appropriate spindle at 30 rpm, MWt of 360,000 Dalton). As the chitosan concentration increases from 1% to 3% this results in a more gradual release of retinoic acid from the chitosan matrix as showvn in FIG. 4.

### Example 6

Preliminary In Vitro Evaluation of Topical Chitosan Delivery System for Retinoids

A. Skin Sample Preparation:

Fresh skin (female abdominal) was obtained from surgery, and upon arrival to the lab was washed and stored with 0.1 M phosphate-buffered saline (PBS) buffer (pH 7.4).

Subcutaneous fat was removed and the skin was rinsed in PBS, it was then dried and stored in the freezer (−20 C.).

Prior to skin splitting, full skin was thawed overnight in sterile PBS. The split skin procedure consisted of taking a 4×4 cm full skin sample and immersing it in water at 60° C. for approximately 60 sec. The epidermis was then carefully removed with forceps and placed on aluminum foil and stored at −20° C. Prior to the permeation experiment, split skin samples were thawed by floatation in water at 22° C. for ˜20-40 minutes.

B. Vehicle Preparation

3.5% HMW-Chitosan (88.8% deacylated chitosan, 1000 cps viscosity, 800,000 MWt; Primex Ingredients SA, Avaldsnes, Norway) was dissolved in 1% acetic acid for 24 hours prior to mixing. The retinoid/chitosan formulation was made up by adding concentrations of retinoids (ATRA or 9-cis-RA) ranging from 0.01% to 0.1% in a colloidal formulation containing 50% ethanol, 1% vitamin E, 8% cremophore RH40, 40% water and 1.75% HMW-chitosan

C. Franz Diffusion Cell Setup

All experiments used 9 mm amberized Franz diffusion cells purchased from PermeGear Inc. (Riegelsville, Pa.). Amberized cells were used to limit light exposure to the retinoic acids. The Franz cells were clamped in series, and water from a water bath (37° C.) was circulated through all cells. A magnetic stirrer was placed underneath all 3 Franz cells to ensure constant agitation of the fluid within the receptor compartment and hence a more homogeneous distribution of the permeant (retinoic acid). Split skin (epidermis) samples of approximately 2.5 cm² surface area were carefully placed upon the receptor compartment (dermis side facing down). The donor cap was then placed upon the skin and carefully clamped into place with a horseshoe clamp.

Receptor fluid (consisting of 25% ethanol and 75% PBS) was placed within the receptor compartment. This concentration of ethanol in PBS prevented the formation of a two-phase system (turbidity) while maintaining the retinoid in solution.

D. Retinoid Percutaneous Studies

A known quantity of conventional retinoid (0.01%-0.1%) was placed in the donor compartment, covered with aluminum foil to prevent evaporation. Samples (200 μl) for spectrophotometric analysis were then removed from the receptor port at timely intervals up to 48 hours and stored in amberized 1 ml Teflon-capped vials. The same quantity of receptor fluid (at 37° C.) was then returned to the receptor compartment to ensure a constant volume. Samples from the vials were diluted five-fold and then quantitated via UV absorbance using a Shimadzu UVI60U spectrophotometer. Maximum absorbance of ATRA (all trans retinoic acid) and 9cRA (9-cis retinoic acid) was at 348.5 nm and 340 nm respectively. The cumulative amount of the applied dose which crossed the epidermis into the receptor chamber was determined as follows: C=R*25/A, where: C=cumulative amount, (μg/cm²); R=retinoid (ag) (from UV reading and standard curve), 25=dilution factor; A=Area of skin exposed to formulation in sample compartment (0.785 cm²).

E. Preliminary Radiolabeled ATRA Percutaneous Studies

Retinoid penetration through human skin was determined as follows:

5μl of 3H-ATPA (NET-1117) were mixed homogenously to 500 μl of HMW-Chitosan to make a 0.001 % gelling solution.consisting of 0.05 g Tretinoin, 50 ml 95% Ethanol, 3.2 g Cremophor RH-40, 1.0 g Vitamin E acetate, 50 ml 2.5% Chitosan (high MW Primex Superior). For the ethanol solution, the chitosan was omitted in the formulation.

200 μl of this solution was then placed on the skin section within the Franz cell. A surface wash was performed at 24 hrs. The skin was washed and blotted and all IVR59 solution, washes and blots placed together in scintillation fluid. The cleaned skin was then dissolved O/N in Soluene 350 and 5 ml scintillation fluid was then added to this solution. An aliquot was removed from the reservoir of the Franz diffusion cell and added to the scintillation fluid (Aquasol-II). All scintillation solutions (top wash, skin and reservoir) were diluted 1:1000 and the radioactivity levels in these samples were counted.

F. Preliminary In Vitro Toxicity and Irritation Studies

The EpiDerm™ Skin Model (Epi-200, MatTek Corporation, Ashland, Mass.) is used to obtain in vitro skin toxicity MTT and IL-1α measurements indicative of skin irritation as follows: Individual human equivalent cultures are transferred to six-well culture plates, each well containing 0.9 ml of culture medium and placed in a humidified incubator at 37° C., 5% CO₂, for 1 hour. Prior to dosing, the medium is replaced with fresh medium. 25 μL of test solution containing 0.05% ATRA with either ethanol or 1.25% IVR59 are topically applied to the apical surface of each culture in duplicate and the culture plate is returned to the incubator.

Culture plates are removed at 18 hrs, according to the protocol. Deionized water is used as the negative control and 0.3% Sodium Dodecyl Sulfate (SDS) as the positive control. The cultures are assayed for residual mitochondrial dehydrogenase enzyme activity (SMTT assay) as an indicator of culture viability (Osborne and Perkins, 1994). Washed cultures are incubated for 3 hrs in a humidified chamber at 37° C. in MTT reagent (Sigma) at a concentration of 1 mg MTT dye per 1.0 ml of incubation medium (EpiDerm™ Assay Medium). The remaining medium was saved for IL-1α cytokine analysis.

At the end of the MTT dye-incubation step, cultures are washed again in PBS and 2 ml of 2-propanol was added to each culture plate to extract the purple formazan product of the MTT dye metabolism. Extraction is performed at room temperature for 2 hrs.

The absorbance of 200 VL aliquots of the formazan/alcohol extracts are measured at 570 nm. The percent viability is calculated using the following formula: % viability=100×[OD(sample)/OD(negative control)].

IL-1α was measured on the saved culture medium using a standard ELISA and protocol from Cayman Chemical Corporation (Ann Arbour, Mich.). The level of absorbance in the 0 pg/ml sample is subtracted from all other standard concentration absorbencies. A linear regression formula for the standard curve was obtained providing the IL-1α concentrations.

The invention has been described in detail with particular references to the preferred embodiments thereof. However, it will be appreciated that modifications and improvements within the spirit and scope of this invention may be made by those skilled in the art upon considering the present disclosure.

The references cited herein are incorporated by reference.

References

Bakkers J, et al. An important developmental role for oligosaccharides during early embryogenesis of cyprinid fish. Proc Natl Acad Sci USA 94:7982-7986, 1997.

Balassa L L. Chitin and Chitin Derivatives for promoting wound healing. U.S. Pat. No. 3,903,268, Sep. 2, 1975.

Balassa L L. Process for Facilitating Wound Healing with N-Acetylated Partially Depolymerized Chitin materials. U.S. Pat. No. 3,914,413. Oct. 21, 1975.

Balassa L L. Process for Promoting wound healing with chitin derivatives. U.S. Pat. No. 3,911,116, Oct. 7, 1975.

Balassa L L. Use of Chitin for Promoting Wound Healing. U.S. Pat. No. 3,632,754, 1972.

Bhawan J, et al. Histologic evaluation of the long-term effects of tretinoin on photodamaged skin, J Dermatol Sci, 11: 177-82, 1996.

Brode G L and Salensky G A. Delivery systems for quaternary and related compounds. U.S. Pat. No. 5,300,494, 1994.

Brown T J, et al.. Absorption of Hyaluronan Applied to the Surface of Intact Skin. J Invest Dermatol 113:740-746, 1999.

Cardinal J R, et al.. Chitosan Compositions for Controlled and Prolongued Release of Macromolecules. U.S. Pat. No. 4,895,724, 1990.

Chandraratna R A S. Rational design of receptor-selective retinoids, J Am Acad Derrnatol, 39:S124-8, 1998.

Chang J, et al.. Interleukin 1 activates phosphate A₂ in rabbit chondrocytes: a possible signal for IL-1 action. J Immunol 136:1283-1287, 1986.

Chensue S W, et al.. Monokine production by Hypersensitive (Schistosoma mansoni EGG) and foreign body (Sephadex bead)-type granuloma macrophages. J Immunol 142:1281-1286, 1989.

Cleenewerk M B, et al. Allergic contact dermatitis due a moisturizing body cream with chitin, Contact Dermatitis, 31:196-197, 1994.

Clifford J L, et al., Mol Endocrinol, 4:1546-1555, 1990.

Curiel-Lewandrowski C, Demierre M. Advances in specific immunotherapy of malignant melanoma. J Am Acad Dermatol, 43:167-185, 2000.

Demierre M, Koh H K. Adjuvant therapy for cutaneous malignant melanoma. J Am Acad Dermatol, 36:747-64, 1997.

Dennis L K. Analysis of the melanoma epidemic, both apparent and real. Arch Dermatol, 135:275-280, 1999.

Egan C L, et al. Cutaneous melanoma risk and phenotypic changes in large congenital nevi: a follow-up study of 46 patients, J Am Acad Dermatol, 39:923-32, 1998.

Fisher G J, et al. The molecular basis of sun-induced premature skin ageing and retinoid antagonism. Nature 379:335-338, 1996.

Fisher G J, et al. Pathophysiology of premature skin aging induced by ultraviolet light. N Engl J Med 337:1419-1428, 1997

Furth J J. The steady-state levels of type I collagen mRNA are reduced in senescent fibroblasts. J Gerontol 46:B 1224-B 1225, 1991

Gallo J M, Hassan E E. Receptor-mediated delivery system. U.S. Pat. No. 5,129,877, 1992.

Gilchrest B A. Treatment of photodamage with topical tretinoin: an overview. J Am Acad Dermatol, 36:S27-S36, 1997.

Gregory C, et al.. Elastin production in human skin fibroblasts cultures and its decline with age. J Invest Dermatol 86:279-285, 1986

Griffiths C E M, et al. Restoration of collagen formation in photodamaged human skin by tretinoin (retinoic acid). N Engl J Med 329:530-534, 1993

Grob J J, et al. Count of benign melanocytic nevi as a major indicator of risk for non-familial nodular and superficial spreading and nodular melanoma. Cancer, 66:387-395, 1990.

Hall H I, et al. Update on the incidence and mortality from melanoma in the United States. J Am Acad Dermatol, 40:35-42, 1999.

Halpern A C, et al. Effects of topical tretinoin on dysplastic nevi. J Clin Oncol, 12:1028-1035, 1994.

Halpern A C. Retinoids and the chemoprevention of melanoma. In: Advances in the biology and treatment of cutaneous melanoma, Boston, Mass., Nov. 6-7, 1998.

Horiuchi Y, Otoyama K. Topical application of 70% deacetylated chitin (DAC-70): a new approach to the management of senile erythroderma following eczema. J Dermatol Treatment 7:97-100, 1996.

Imai T, et al., Int. J. Pharm., 67:11-20, 1991.

Johnson B D, et al. Effects of donor age on protein and collagen synthesis in vitro by human diploid fibroblasts. Lab Invest 55:490-496, 1986

Kang S, et al., Application of retinol to human skin in vivo induces epidermal hyperplasia and cellular retinoid-binding proteins characteristic of retinoic acid but without measurable retinoic acid levels or irritation. J Invest Dermatol 105: 549-556, 1995

Kligman A M. The growing importance of topical retinoids in clinical dermatology: a retrospective and prospective analysis. J Am Acad Dermatol 39:S2-S7, 1998.

Koh H K, Geller A C. Public health interventions for melanoma: prevention, early detection, education. Hematology/Oncology Clinics of North Americ,12:903-928, 1998.

Koh H K. Cutaneous melanoma. N Engl J Med, 325:171-182, 1991.

Kristl J, et al. Int. J. Pharm., 99:13-19, 1993

Labrecque D, et al. Novel Bis-styrl Analogs with Retinoidal Activity, J Med Chemistry, in press.

Lavker P M. Cutaneous aging: chronologic versus photoaging. In: Gilchrest B A (ed.). Photoaging. Cambridge, Mass.: Blackwell Science, pp 123-135, 1995

Lavker R M. Structural alterations in exposed and unexposed aged skin. J Invest Dermatol 73:559-566, 1979

Lehman P A, et al. “Percutaneous Absorption of Retinoids: Influence of Vehicle, Light Exposure, and Dose”, J Invest Dermatol, 91:56-61, 1988.

Lotan R. Retinoids in cancer chemoprevention. FASEB J. 10: 1031-9, 1996.

Maekawa A, Wada M. Food containing chitin or its derivatives for reduction of blood and urine uric acid. JP 03,280,852, 1990.

Mansell P W A. Polysaccharides in skin care. Cosmet Toilet 109:67, 1994.

Mays P K, et al. Similar age-related alterations in collagen metabolism in rat tissues in vivo and fibroblasts in vitro. Biochem Soc Trans 18:957, 1990

Meier F. et al. Molecular events in melanoma development and progression, Front Biosci, 3:D 1005-10, 1998.

Mills O H and Berger R S. Irritation potential of a new topical tretinoin formulation and a commercially-available tretinoin formulation as measured by patch testing in human subjects, J Am Acad Dermatol, 38:S11-6, 1998.

Mita N, et al. Pharnaceuticals and food containing edible fibers of chitosan for hyperuricemia control. JP 02,311,421, 1989.

Muzzarelli R, et al., Biomaterials, 9:247-252, 1988.

Muzzarelli R A A, et al. (eds) Chitin in nature and technology, Plenum Press, New York, 1986.

Muzzarelli R A A, et al., histology and clinical uses of chitins and chitosans in wound healing. In: Chitin and Chitinases, Jolles P and Muzzarelli RAA (Eds), Birkhauser Verlag Publ., Basel, Switzerland, pp.251-264, 1999.

Muzzarelli R A A. Biochemical significance of exogenous chitins and chitosans in animals and patients. Carbohydr Polym 20:7-16, 1993.

Muzzarelli R A A. Wound dressing materials. In: The polymeric materials encyclopedia. CRC Press, Boca Raton, Fla., 1996.

Nagpal S and Chandraratna A S. Retinoids as Anti-Cancer Agents, Curr Pharm Design, 2:295-316, 1996.

Neudecker B A, et al. Hyaluronan: The Natural Skin Moisturizer. In: Cosmeceuticals:, Drugs vs Cosmetics, Elsner P. and Maibach H I (Eds), Marcel Dekker, Inc., New York, N.Y., pp.319-355, 2000

Nishimura K, et al. “Stimulation of cytokine production in mice using deacetylated chitin” Vaccine 4:151-156, 1986.

Nishimura K, et al. “Adjuvant activity of chitin derivatives in mice and guinea-pig”, Vaccine3:379-384, 1985.

Nishimura K, et al. “Immunological activity of chitin and its derivatives”, Vaccine 2:93-99, 1984.

Nishimura K, et al. Mol. Biother., 2:115-120, 1990.

Otterlei M, et al., Vaccine, 12:25-32, 1994.

Papineau A M, et al., Food Biotechnol., 5:45-47, 1991.

Partain E M, Brode G L, Delivery Systems for Pharmaceutical or Therapeutic Actives. U.S. Pat. No. 4,946,870, 1990.

Partain E M, Brode G L. Acid Decrystallization of Aminopolysaccharides and Derivatives Thereof. U.S. Pat. No. 4,929,722, 1990.

Quigley J W and Bucks D A W. Reduced skin irritation with tretinoin containing polyolprepolymer-2, a new topical tretinoin delivery system: A summary of preclinical and clinical investigations, J Am Acad Dermat, 38:S5-10, 1998.

Sachetto J P. “A substitute for hyaluronic acid for cosmetics application. 14^{th} IFSCC Congress, 17-19 September 1986, Barcelona, Spain, 2:867-877, 1986.

Saiki I, et al., In: Chitin Derivatiues in Life Sciences, Tokura S and Azuma I (Eds), Chiba, Japan, pp.20-6, 1992.

Sankaranarayanan R, Mathew B. Retinoids as cancer-preventive agents. IARC Sdc Pubt 139:47-59, 1996.

Sauter E R, Herlyn M. Molecular biology of human melanoma development and progression. Molecular Carcinogenesis, 23:132-43, 1998 Seaton T L, In:Applied Therapeutics: the clinical use of drugs, Young L Y, Koda-Kimble M A (eds), 6th ed., Vancouver, Was. Applied Therapeutics, Inc., p.37, 1995.

Semino C E, et al. Homologs of the Xenopus developmental gene DG42 are present in zebrafish and mouse and are involved in the synthesis of Nod-like chitin oligosaccharides during early embryogenesis. Proc Natl. Acad. Sci. USA 93:4548-4553, 1996.

Shibata Y, et al. Alveolar macrophage priming by intravenous administration of chitin particles, polymers of N-acetyl-D-glucosamine, in mice. Infect Immun 65:1734-1741, 1997.

Siegenthaler G, et al. Topical Retinaldehyde on Human skin: Clinical and Biological Observations, In: Retinoids: From Basic Science to Clinical Applications, M. A. Livrea and G. Vidali (eds), Birkhauser Verlag, Basel, Switzerland, pp. 329-335, 1994.

Smith J G, et al. Alterations in human dermal connective tissue with age and chronic sun damage. J Invest Dermatol 39:347-356, 1962

Spanjaard R A, et al. Specific activation of retinoic acid receptors (RARs) and retinoid X receptors (RXRs) reveals a unique role for RARγ in differentiation and apoptosis of S91 melanoma cells. J Biol Chem, 272:18990-18999, 1997.

Stan-Posthuma J J, et al. Effect of topical tretinoin under occlusion on atypical naevi, Melanoma Research, 8:539-48, 1998.

Suzuki K. A new drug delivery system for local cancer chemotherapy using cisplatin and chitin. Anticancer Reseach; 15:423-426, 1995.

Talwar H, et al. Reduced type I and type III procollagens in photodamaged adult human skin. J Invest Dermatol 105:285-291, 1995

Tokura S and Azuma I. (Eds)In: Chitin Deriuaties in life sciences. Japan Soc. Chitin, Sapporo, Japan, 1992,

Tucker M A, et al. D 4th, Clark W H Jr. Clinically recognized dysplastic nevi. A central risk factor for cutaneous melanoma, JAMA, 277:1439-44, 1997.

Vanlerberghe G, Sebag H. Cosmetic compositions for the skin containing a chitosan derivative. U.S. Pat. No. 3,953,608, 1976.

Varani J, et al. Vitamin A Antagonizes Decreased Cell Growth and Elevated Collagen-Degrading Matrix Metalloproteinases and Stimulates Collagen Accumulation in Naturally Aged Human Skin. J Invest Dermatol 114:480-486, 2000.

Varki A. Does DG42 synthesize hyaluronan or chitin? A controversy about oligosaccharides in vertebrate development. Proc Natl Acad Sci USA. 93:4523-4525, 1996.

Wlada M. Effect of chitin, chitosan intake on metabolism of uric acid. GekkanFudo Kemikaru 11:25-31, 1995.

Watanabe K, et al., Carbohydr. Polym., 17:29-37, 1992.

West M D. The cellular and molecular biology of skin aging. Arch Dermatol 130:87-92, 1994

Wingo P A, et al. Annual report to the nation on the status of cancer, 1973-1996, with a special section on lung cancer and tobacco smoking. J Natl Cancer Inst; 91:675-90, 1999.

Won R, et al. Methods and Compositions for Topical Delivery of Retinoic Acid. U.S. Pat. No. 5,955,109, Sep. 21, 1999

Won R. Two step method for preparation of controlled release formulations. U.S. Pat. No. 5,145,675, Sep. 8, 1992.

We claim:

1. A carrier base for the topical delivery of an active agent comprising a high viscosity chitosan biopolymer.

2. The carrier base according to claim 1, wherein the chitosan has a molecular weight of at least about 100,000 Dalton.

3. The carrier base according to claim 1, wherein the chitosan has a concentration of at least about 2 weight % .

4. A composition for the topical delivery of an active agent comprising a carrier base according to claim 1 and an active agent.

5. The composition according to claim 4, wherein the active agent comprises a pharmaceutical active.

6. The composition according to claim 5, wherein the pharmaceutical active is used for the treatment of skin diseases.

7. The composition according to claim 6, wherein the pharmaceutical active is selected from retinoids, corticosteroids, non-steroidal anti-inflammatory drugs (NSAIDS), hormones, anti-fungal agents, anti-septic agents, local anaesthetics, kerolytic agents, and 5-FU.

8. The composition according to claim 4, wherein the active agent comprises a therapeutic active.

9. The composition according to claim 8, wherein the therapeutic active comprises vitamins and alpha-hydroxy acids.

10. The composition according to claim 4, further comprising at least one additional active agent.

11. The composition according to claim 4, wherein the chitosan has a molecular weight of at least about 100,000 Daltons.

12. The composition according to claim 11, wherein the chitosan in present in a concentration of up to about 3%.

13. The composition according to claim 4, wherein the chitosan has a molecular weight of about 10,000 to about 250,000 Daltons.

14. The composition according to claim 13, wherein the chitosan is present in a concentration of up to about 8%.

15. A composition for the topical delivery of retinoids comprising a carrier base and a retinoid, wherein the carrier base comprises a high viscosity chitosan biopolymer.

16. The composition according to claim 15, wherein the chitosan biopolymer has a molecular weight of at least 100,000 Dalton and at a concentration of at least 2 weight %.

17. The composition according to claim 16, which is in a gel.

18. The composition according to claim 16, which is in a cream.

19. The composition according to claim 16, which is a lotion.

20. A method of controlling the release of an active agent from a carrier, comprising:
providing as a carrier base a high viscosity chitosan biopolymer;
providing the active agent; and
mixing the active agent and the chitosan.

21. The method according to claim 20, wherein the chitosan biopolymer has a molecular weight of at least 100,000 Dalton and at a concentration of at least 2 weight %

22. The method according to claim 20, wherein the active agent comprises a pharmaceutical active.

23. The method according to claim 22, wherein the pharmaceutical active is used for the treatment of skin diseases.

24. The method according to claim 22, wherein the pharmaceutical active is selected from retinoids, corticosteroids, non-steroidal anti-inflammatory drugs (NSAIDS), hormones, anti-fungal agents, anti-septic agents, local anaesthetics, kerolytic agents, and 5-FU.

25. The method according to claim 20, wherein the active agent comprises a therapeutic active.

26. The method according to claim 25, wherein the therapeutic active comprises vitamins and alpha-hydroxy acids.

27. A method of treating skin diseases comprising providing to the diseased skin a carrier base containing a high viscosity chitosan biopolymer and an active agent.

28. The method according to claim 27, wherein the chitosan has a molecular weight of at least 100,000 Dalton.

29. The method according to claim 27, wherein the chitosan is at a concentration of at least 2 weight %.

30. The method according to claim 27, wherein the skin disease comprises acne, melanoma, premature aging, photodamage.

31. The method of treating skin diseases according to claim 27, further providing an anti-cancer drug.
